(19) United States
(12) Patent Application Publication
(10) Pub. No.: US 2020/0080155 A1
(43) Pub. Date: Mar. 12, 2020

(54) DOT1L INHIBITORS AND USES THEREOF

(71) Applicant: Baylor College of Medicine, Houston, TX (US)

(72) Inventors: Rachel RAU, Houston, TX (US); Margaret A. GOODELL, Houston, TX (US)

(21) Appl. No.: 16/441,381

(22) Filed: Jun. 14, 2019

Related U.S. Application Data
(63) Continuation of application No. 15/502,623, filed on Feb. 8, 2017, now abandoned.
(60) Provisional application No. 62/035,373, filed on Aug. 8, 2014.

(51) Int. Cl. C12Q 1/6886; A61K 31/52; A61K 31/519; A61K 31/00; C07H 19/16

(52) U.S. Cl. CPC C12Q 1/6886; A61K 31/52; A61K 31/519; A61K 31/00; C12Q 2600/106; C07H 19/16; C12Q 2600/156; C12Q 2600/154; C12Y 201/01037

(57) ABSTRACT

The present disclosure relates to methods of treating AML associated with DNMT3A mutations by administering one or more DOT1L inhibitors or related pharmaceutical compositions to subjects in need thereof

### RELATED APPLICATIONS

This application is a continuation of U.S. application Ser. No. 15/502,623, filed Feb. 8, 2017, which is a U.S. National Phase application, filed under 35 U.S.C. § 371, of International Application No. PCT/US2015/044394, filed Aug. 8, 2015, which claims the benefit under 35 U.S.C. § 119(e) of U.S. provisional application U.S. Ser. No. 62/035,373, filed Aug. 8, 2014, and entitled “DOT1L Inhibitors and Uses Thereof”, the entire contents of each of which are incorporated herein by reference in their entireties.

### FEDERALLY SPONSORED RESEARCH

This invention was made with support from government grant number NCI/NIH K12 CA090433-11 awarded by the National Institutes of Health and grant number DK092883-01 awarded by the National Institutes of Health. The government has certain rights in the invention.

### INCORPORATION-BY-REFERENCE OF SEQUENCE LISTING

The contents of the text file named “EPIZ-064-N01US_ST25.txt”, which was created on Jul. 7, 2017 and is 7.99 KB in size, are hereby incorporated by reference in their entireties.

### BACKGROUND

The genetic analysis of samples from patients with acute myeloid leukemia (AML) has identified a range of somatic mutations, including certain mutations in the DNA (cytosine-5)-methyltransferase 3A (DNMT3A) gene. DNMT3A is a DNA methyltransferase that catalyzes methylation of CpG sequences in DNA. Mutations of the DNA methyltransferase DNMT3A are often associated with poor prognosis in AML patients.

### SUMMARY

Aspects of the disclosure relate to methods and compositions for treating acute myeloid leukemia (AML) and other haem malignancies associated with mutations in DNA methyltransferase 3A (DNMT3A). Aspects of the disclosure are based, at least in part, on the determination that AML associated with one or more mutations in DNMT3A is responsive to the inhibition of DOT1L activity. Accordingly, in some embodiments, a subject having AML associated with one or more mutations in DNMT3A can be treated with one or more DOT1L inhibitor compounds as described herein. In some embodiments, a subject diagnosed with AML and having expanded methylation canyons associated with DNMT3A mutation can be treated with one or more DOT1L inhibitor compounds as described herein. In some embodiments, a subject having expanded methylation canyons associated with DNMT3A comprising HOX gene clusters can be treated with one or more DOT1L inhibitor compounds as described herein.

Accordingly, aspects of the disclosure provide methods and compositions for the treatment of AML. In some embodiments, aspects of the disclosure are useful to identify AML patients that are responsive to treatment with one or more DOT1L inhibitor compounds. In some embodiments, a subject having one or more clinical symptoms, gene expression markers, and/or other indicia of AML associated with mutation(s) in DNMT3A is identified as a candidate for treatment with a DOT1L inhibitor compound (e.g., as a subject in need of treatment with a DOT1L inhibitor compound). In some embodiments, the subject is treated with one or more DOT1L inhibitor compounds as described herein.

In some embodiments, a subject at risk of developing AML associated with mutation(s) in DNMT3A can be treated with one or more DOT1L inhibitor compounds to prevent or slow the progression of the disease.

Non-limiting examples of DOT1L inhibitor compounds include a compound of formula:

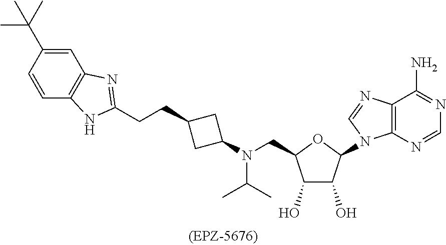 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof, and a compound of formula:

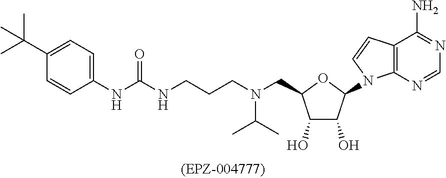 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof.

In some embodiments, a DOT1L inhibitor is a compound of formula:

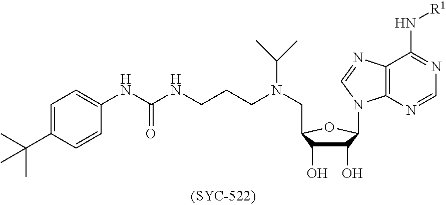

wherein R1 is a H, or a pharmaceutically acceptable salt, prodrug, metabolite, polymorph, solvate or stereoisomer thereof. However, it should be appreciated that other DOT1L inhibitors as described herein can be used.

Accordingly, the present disclosure provides methods and compositions for treating, preventing, and/or alleviating one or more symptoms of certain AMLs by administering to a subject in need thereof a therapeutically effective amount of a DOT1L inhibitor.

In some embodiments, a subject having AML associated with one or more mutations in DNMT3A can be identified by assaying a subject having AML (e.g., a biological sample obtained from the subject having AML) to determine whether one or more markers (e.g., genetic markers) of DNMT3A mutation(s) are present. In some embodiments, the presence of one or more dominant negative mutations in at least one allele of DNMT3A are detected in the subject (e.g., in a biological sample obtained from the subject). In some embodiments, the presence of one or more loss of function mutations (e.g., deletions or other loss of function mutations) in both alleles of DNMT3A are detected in the subject (e.g., in a biological sample obtained from the subject).

In adult AML, 20-25% of patients have DNMT3A mutations. Approximately 60% of these mutations are predicted to affect the R882 position that lies within the methyltransferase domain. Mutations affecting the R882 position result in a dominant negative loss of function (Russler-Germaine 2013, Kim 2013) and are almost exclusively heterozygous. The remainder of DNMT3A mutations are predicted to affect other amino acids (non-R882 mutations). Approximately 5-10% of patients with non-R882 mutations have either homozygous mutations or two different DNMT3A mutations. Accordingly, in some aspects, methods and compositions described herein can be used to treat subjects having AML associated with one or two DNMT3A mutations.

Hereditary mutations of DNMT3A have only recently been described in patients with an overgrowth syndrome with intellectual disability (Tatton-Brown Nat Genet. 2014). In some aspects, methods and compositions described herein can be used to treat subjects having AML associated with hereditary DNMT3A mutations.

In some aspects, a subject having AML associated with a DNMT3A mutation has one or more expanded methylation canyons (e.g., the length of one or more hypomethylated DNA regions is expanded). In some embodiments, an expanded methylation canyon encompasses a genomic region encoding transcription factors. In some embodiments, an expanded methylation canyon encompasses a genomic region encoding genes frequently dysregulated in human leukemia. In some embodiments, an expanded methylation canyon comprises HOX gene clusters (e.g., HOXA and/or HOXB gene clusters).

In some embodiments, the expanded methylation canyons associated with DNMT3A mutation are coated with H3K79me2. In some embodiments, a subject having AML associated with one or more mutations in DNMT3A can be identified by assaying a subject having AML (e.g., a biological sample obtained from a subject having AML) to determine whether increased levels of H3K79me2 are present in the sample. In some embodiments, a general increase in H3K79me2 can be detected (e.g., in a sample from the subject). In some embodiments, an increase in H3K79me2 associated with an expanded methylation canyon can be detected (e.g., in a sample from the subject). In some embodiments, an increased level of H3K79me2 is an amount that is higher than expected in a healthy sample. For example, in some embodiments an increased level is an amount that is higher than in a DNMT3A wild-type control (for example an amount in a sample from a subject that does not have a DNMT3A mutation, or an amount in a cell line that does not have a DNMT3A mutation). In some embodiments, a level of H3K79me2 in a sample can be compared to a reference amount (e.g., an amount previously measured for a DNMT3A wild-type control).

In some embodiments, the present disclosure provides a method for treating, preventing, and/or alleviating one or more symptoms of AML in a subject comprising: obtaining a sample from the subject and detecting one or more mutations in DNMT3A in the sample, wherein the presence of one or more mutations in DNMT3A indicates the subject is responsive to a DOT1L inhibitor. In some embodiments, one or more DOT1L inhibitor compounds are administered to the subject in a therapeutically effective amount.

In some embodiments, the present disclosure provides a method for treating, preventing, and/or alleviating one or more symptoms of AML in a subject comprising: obtaining a sample from the subject; detecting the presence of one or more expanded methylation canyons associated with DNMT3A in the sample; and administering to the subject a therapeutically effective amount of one or more DOT1L inhibitors when said canyons are present in the sample.

In any of the methods described herein, the sample can be selected from bone marrow, peripheral blood cells, blood, cerebrospinal fluid, skin lesions, chloroma biopsy, plasma, serum, urine, saliva, a cell, or other suitable source.

In another aspect, the disclosure provides methods of selecting a therapy for a subject having leukemia (e.g., AML). In some embodiments, a method includes detecting the presence of (a) one or more mutations in DNMT3A, and/or (b) one or more expanded methylation canyons associated with DNMT3A in a sample from the subject; and selecting, based on the presence of (a) and/or (b) in the sample, a DOT1L inhibitor for treating leukemia. Accordingly, in some embodiments, a method includes detecting the presence of one or more mutations in DNMT3A in a sample from the subject, or detecting one or more expanded methylation canyons associated with DNMT3A in a sample from the subject, or detecting the presence of one or more mutations in DNMT3A and one or more expanded methylation canyons associated with DNMT3A in a sample from the subject; and selecting, based on the presence of any of the foregoing in the sample, a DOT1L inhibitor for treating leukemia. In some embodiments, the method further includes administering to the subject a therapeutically effective amount of the DOT1L inhibitor.

In some aspects, a therapeutically effective amount of one or more DOT1L inhibitor compounds can be formulated with a pharmaceutically acceptable carrier for administration to a mammal, for example a human subject, for use in treating or preventing leukemia (e.g., AML) associated with one or more mutations in DNMT3A, and/or one or more expanded methylation canyons associated with DNMT3A mutation.

Accordingly, in certain embodiments, the compounds of the present disclosure are useful for treating, preventing, or reducing the risk of leukemia (e.g., AML) or for the manufacture of a medicament for treating, preventing, or reducing the risk of leukemia (e.g., AML). In some embodiments, compounds or formulations described herein can be administered, for example, via oral, parenteral, otic, ophthalmic, nasal, or topical routes, to provide an effective amount of the compound to the mammal. In some embodiments, compounds or formulations described herein can be administered, for example, via intravenous (IV) infusion (e.g., continuous IV infusion for several days to several weeks).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing aspects of the present disclosure, suitable methods and materials are described herein. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the disclosure will be apparent from the following detailed description and claims.

### BRIEF DESCRIPTION OF THE DRAWINGS

FIG. 1 shows a pie chart illustrating methylation canyon size dynamics in Dnmt3a knock-out mouse Hematopoietic Stem Cells (HSC). On 44% of canyons, the edges were eroded such that they increased in size, and 31% of canyons experienced hypermethylation at the edges, such they decreased in size, and 25% experienced no significant change in size.

FIG. 2 illustrates an embodiment where H3K79me2 is specifically associated with expanding methylation canyons of the HoxA gene cluster. Canyon expansion is a result of Dnmt3a loss.

FIG. 3 illustrates an embodiment where H3K79me2 is specifically associated with expanding methylation canyons of the HoxB gene cluster. Canyon expansion is a result of Dnmt3a loss.

FIG. 4 illustrates an embodiment of reduction of cell proliferation (top) and induction of apoptosis (bottom) of patient-derived AML cell lines in response to treatment with the DOT1L inhibitor SYC-522. Note that AML cell lines respond to SYC-522 treatment in a dose-dependent fashion. OCI AML2 and OCI AML3 cells bear mutations in DNMT3a (homozygous R635W and heterozygous R882C, respectively). MV4-11 is an MLL-rearranged cell line known to be sensitive to DOT1L inhibition.

FIG. 5 illustrates an embodiment where treatment of DNMT3A mutant AML cell lines OCI AML2 and OCI AML3 with DOT1L inhibitor SYC-522 induces apoptosis (top) and terminal differentiation (bottom) in a dose-dependent manner. Induction of terminal differentiation was measured by staining with CD14, a marker of cell differentiation.

FIG. 6 illustrates an embodiment where treatment of DNMT3A mutant AML cell lines OCI AML2 and OCI AML3 with the DOT1L inhibitor SYC-522 causes a reduction in H3K79 methylation in a time-dependent manner.

FIG. 7 shows a reduction in cell proliferation resulting from treatment with the DOT1L inhibitor EPZ004777 in the DNMT3A mutant AML cell lines (OCIAML2, OCIAML3(line 1) and OCIAML3 (line 2)). A significant decrease in cell proliferation was observed in all cell lines treated with 3 μM of the compound.

FIG. 8 illustrates an embodiment where apoptosis of DNMT3A mutant human AML cells is caused by treatment with 3 μM of EPZ00477. The top panel shows % of apoptotic cells marked by Annexin V binding above baseline in OCIAML2, OCIAML3(line 1) and OCIAML3 (line 2) cells. The bottom panel shows representative flow cytometry data indicating the increase in Annexin V binding after 14 days of treatment (right peak) above untreated baseline (left peak).

FIG. 9 shows flow cytometry data of OCIAML2 and OCIAML3 cells treated with 3 μM EPZ004777 and stained for CD14, a marker of cell differentiation. A significant increase in CD14-positive cells was observed in treated groups by Day 7 and Day 9 for both cell lines.

FIG. 10 illustrates an embodiment where DOT1L inhibition reduces HOX gene expression in DNMT3A mutant cells. Bars represent from left to right: untreated, day 6, and day 10 of treatment with 3 μM EPZ004777.

FIGS. 11A-11E show an embodiment where Dnmt3a^{-/-} HSCs are characterized by increased DOT1L expression and increased H3K79 methylation that is associated with altered DNA methylation. FIG. 11A shows RNAseq data showing mRNA expression of DOT1L in murine Dnmt3aKO HSCs (bottom panel) compared to HSC from wild-type HSCs from 4-, 12- and 24-month old mice. FIG. 11B shows quantified DOT1L RNAseq data. FIG. 11C shows relative density of H3K79me2 at transcription start sites (TSS), protein coding start sites, undermethylated regions (UMRs) and DNA methylation canyons in Dnmt3aKO HSCs relative to wild-type HSCs. FIG. 11D shows relative H3K79me2 density at DNA methylation canyons that expand with Dnmt3a deletion (Dnmt3aKO HSCs; wild-type HSCs), canyons that align (Dnmt3aKO HSCs; wild-type HSCs), and canyons that shrink with Dnmt3a deletion (Dnmt3aKO HSCs; wild-type HSCs). FIG. 11E shows a representative DNA methylation canyon that expands with Dnmt3a deletion (DNA methylation; Canyon extended bar) and associated H3K79me2 in wild-type HSCs and Dnmt3aKO HSCs.

FIGS. 12A-12C illustrate an embodiment where DOT1L-induced histone 3 lysine 79 methylation is increased in DNMT3A-mutant human AML cell lines. FIG. 12A shows relative level of unmethylated H3 lysine 79. FIG. 12B shows relative level of mono-methylated H3K79. FIG. 12C shows relative level of di-methylated H3K79. Relative levels were measured by mass spectrometry in four AML cell lines KG1, THP1, OCI AML2 and OCI AML3.

FIGS. 13A-13C show an embodiment where pharmacologic DOT1L inhibition reduces cellular H3K79me and oncogenic HOX gene expression in DNMT 3A mutant AML cells. FIG. 13A shows immunoblot analysis of cellular H3K79me2 in OCI AML2 and OCI AML3 cells after treatment with 3 μM EPZ004777. FIG. 13B shows relative expression determined by quantitative RT PCR of leukemogenic HOXA9, MEIS1, HOXB3, HOXB8 genes and the housekeeping gene GAPDH in OCI AML 2 cells treated with 3 μM EPZ004777 or vehicle control. FIG. 13C shows relative expression determined by quantitative RT PCR of leukemogenic HOXA9, MEIS1, HOXB3, HOXB8 genes and the housekeeping gene GAPDH in OCI AML3 cells treated with 3 μM EPZ004777 or vehicle control.

FIGS. 14A-14B show an embodiment where EPZ004777 treatment inhibits the proliferation of DNMT3A-mutant human AML cell lines in a dose- and time-dependent fashion. FIG. 14A shows growth of OCI AML2 (left panel) and OCI AML3(right panel) cells treated with increasing concentrations of EPZ004777 for 14 days. Numbers are plotted on logarithmic scale. FIG. 14B shows HL60, MV411, OCI AML2 and OCI AML3 cells treated with 3 μM EPZ004777 or vehicle control. Cells were re-plated at a constant concentration in fresh drug-containing media every 2-3 days.

FIGS. 15A-15E show one embodiment where EPZ004777 treatment induces apoptosis, cell cycle arrest and terminal differentiation in DNMT3A-mutant human AML cells in a dose-dependent manner. OCI AML2 and OCI AML3 cells were treated with increasing concentrations of EPZ004777 or vehicle control. Cells were re-plated at a constant concentration in fresh drug-containing media every 2-3 days. HL60, MV411, OCI AML2 and OCI AML3 cells were treated with 3 μM EPZ004777 or vehicle control. Cells were re-plated at a constant concentration in fresh drug-containing media every 2-3 days. FIG. 15A shows induction of apoptosis as measured every 2-3 days by Annexin V binding (AVB) flow cytometry assay of cells treated with increasing concentrations of EPZ004777. Flow histograms on days 5, 11 and 14 of treatment are shown in the left panels and quantification of % of cells AVB+ is shown in the right panels. FIG. 15B shows time dependent induction of apoptosis as measured by AVB flow cytometry assay shown as percentage of cells AVB+ minus percentage of AVB+ cells treated with vehicle control. FIG. 15C shows quantification of cell cycle analyses of OCI AML2 and OCI AML3 cells treated with increasing concentrations of EPZ004777 or vehicle control performed by flow cytometry for DNA content at specified time points. FIG. 15D shows representative flow cytometry plots of PI DNA content cell cycle analysis for OCI AML2 cells and OCI AML3 cells (left panels) with quantification in the graph on the right. FIG. 15E shows representative flow plots of CD14 cell surface expression OCI AML 2 and OCI AML 3 cells treated with vehicle control (untreated) or 3 μM EPZ004777 for 14 days (left panels) and quantification of percentage of CD14⁺0 cells treated with 3 μM EPZ004777 or vehicle control at specified time points. Error bars represent standard deviation.

FIGS. 16A-16C illustrate in vivo efficacy of pharmacologic DOT1L inhibition in DNMT3A-mutant AML. H3K79me2 levels as measured by ELISA measured in OCI AML3 subcutaneous tumors (left) and bone marrow from vehicle control-treated animals or animals treated with 35 or 70 mg/kg/day EPZ5676 administered via continuous IV infusion (right). FIG. 16B shows volume of OCI AML3 subcutaneous tumors over time in vehicle control treated animals and animals treated with 35 or 70 mg/kg/day EPZ5676 administered via continuous IV infusion for 21 days. FIG. 16C shows relative expression of MEIS1 and HOXB3 in OCI AML3 subcutaneous tumors in vehicle control treated mice and mice treated with 35 or 70 mg/kg/day EPZ5676 for 21 days.

FIGS. 17A-17D show an embodiment where DOT1L inhibitor treatment selectively inhibits the in vitro growth and induces terminal differentiation of primary patient samples with DNMT3A mutations. FIG. 17A shows relative colony forming units (CFU) of normal cord blood CD34+ cells and primary AML samples wild-type for both DNMT3A and MLL (left panel), primary AML samples with MLL anomalies (middle panel), and primary AML samples with DNMT3A mutations (right panel) treated with vehicle control or 3 μM EPZ004777. Assays performed in triplicate, error bars represent standard deviation. Patient numbers on horizontal axis correspond to patient numbers in Table 1. FIG. 17B shows average change in CFC of primary patient samples treated with EPZ004777 compared to vehicle treated control. FIG. 17C shows flow cytometry analysis of CD14 expression of primary AML cells with DNMT3A mutation isolated from plates after treatment with vehicle control or 3 μM EPZ004777. FIG. 17D shows H&E staining of primary AML cells with DNMT3A mutation isolated from plates after treatment with vehicle control or 3 μM EPZ004777.

FIGS. 18A-18B illustrate an embodiment where SYC-522 inhibits growth and induces apoptosis of DNMT3A-mutant AML cell lines in a dose-dependent manner. OCI AML2 and OCI AML3 cells were treated with increasing concentrations of SYC-522 or vehicle control. Cells were re-plated at a constant concentration in fresh drug-containing media every 3-4 days. FIG. 18A shows growth curves of cells treated with SYC-522 or vehicle control for 10 days. Numbers are plotted on logarithmic scale. FIG. 18B shows induction of apoptosis as measured on days 7 and 10 of treatment with SYC-522 or vehicle control by Annexin V binding (AVB) flow cytometry assay.

FIG. 19 shows an embodiment where EPZ004777 treatment of primary AML patient samples (patient numbers corresponding to patient numbers shown in Table 1) with DNMT3A mutation induces terminal differentiation. Flow cytometry plots of primary AML primary patient samples isolated from culture plates after treatment in CFC assay with vehicle control (untreated) or 3 μM EPZ004777 are shown. Plots show live (PI negative) CD45+ cells.

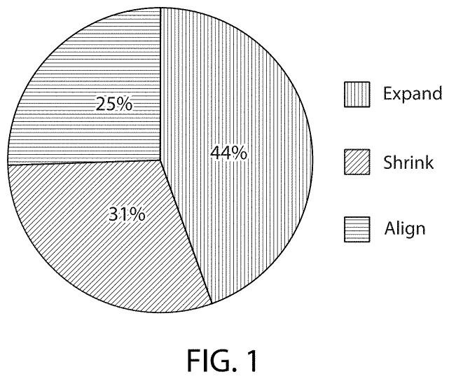

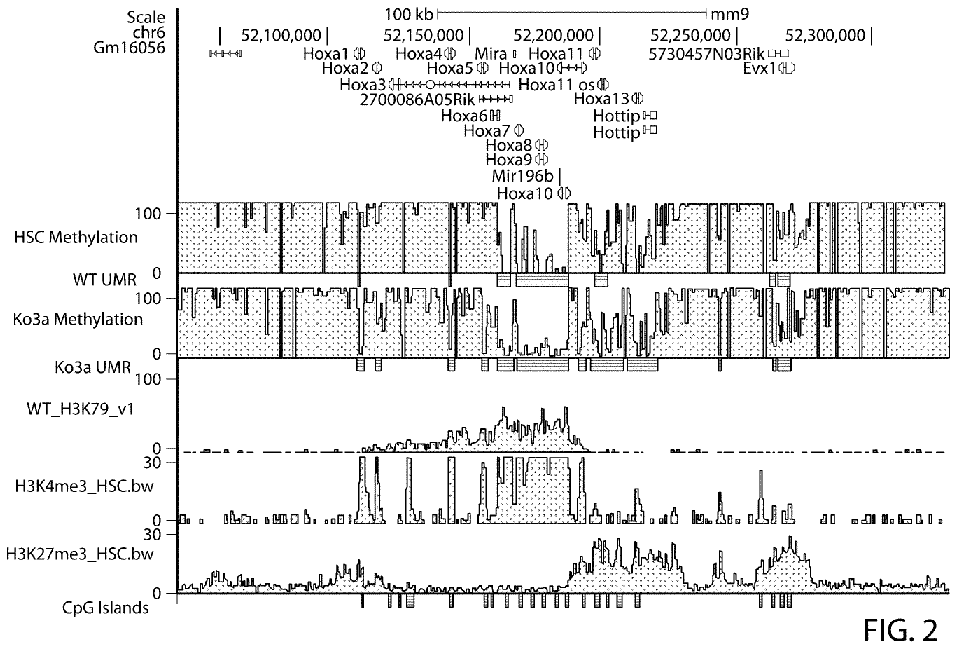

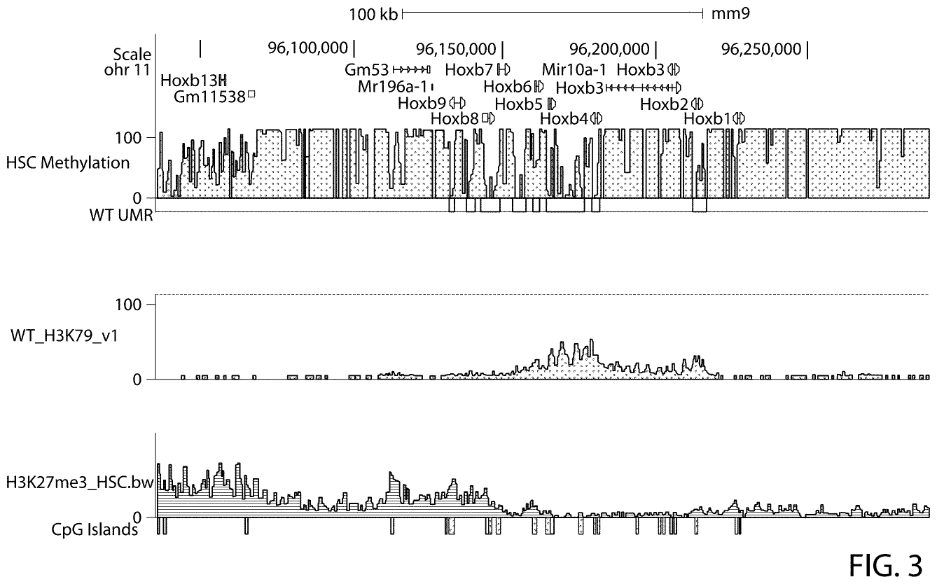

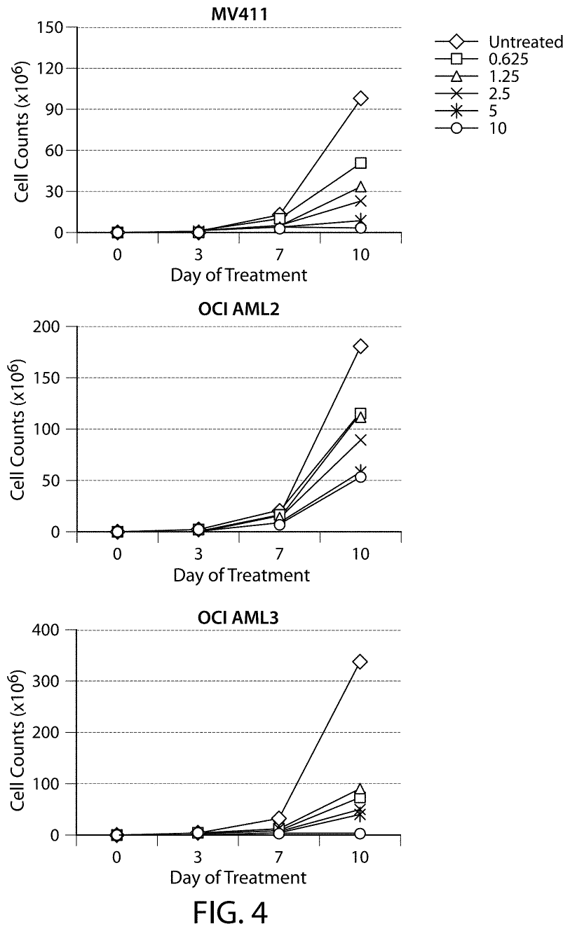

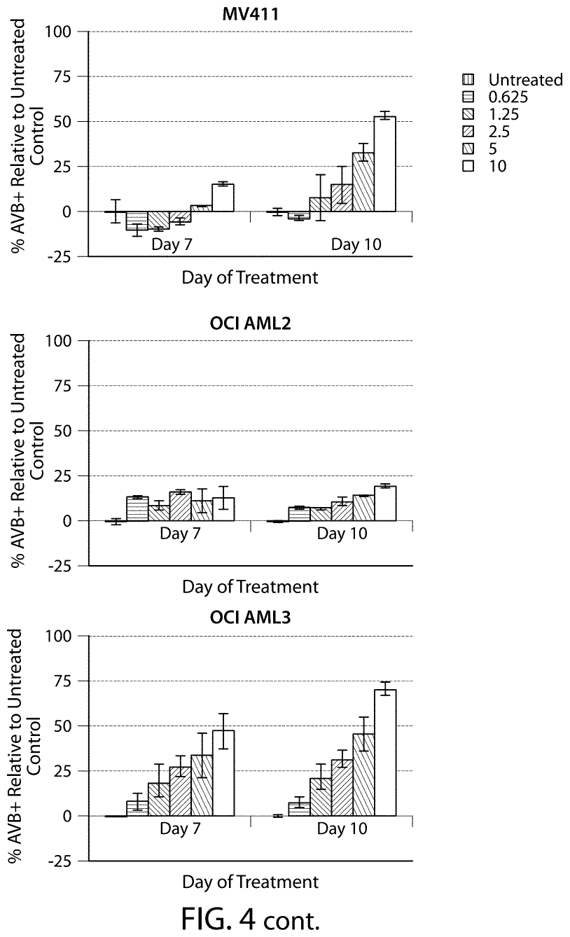

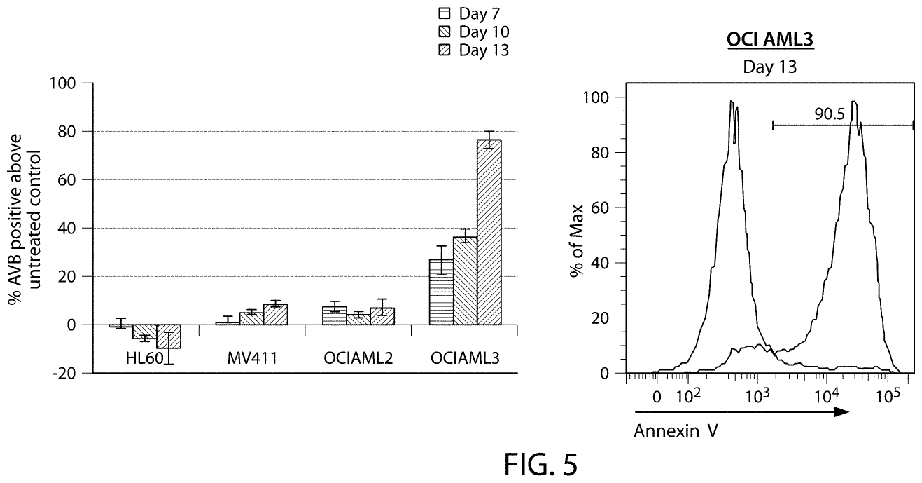

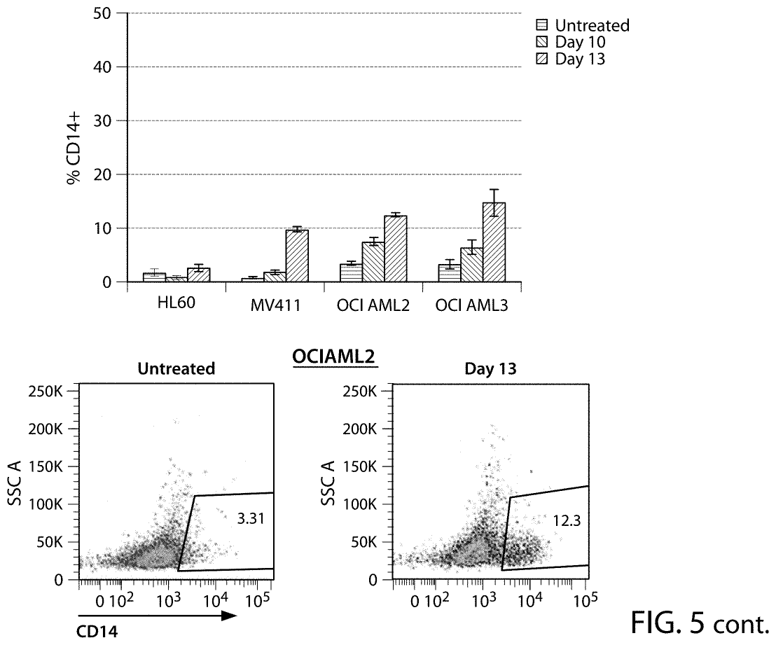

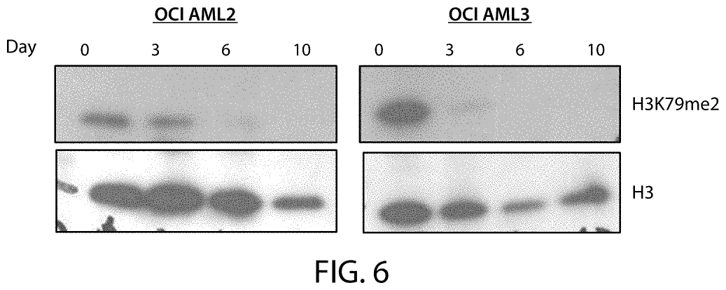

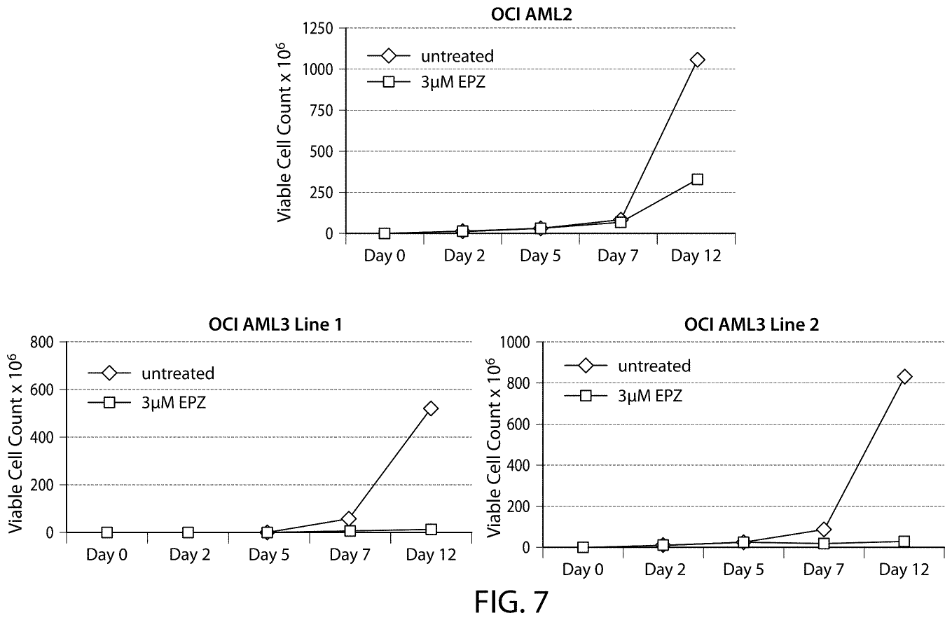

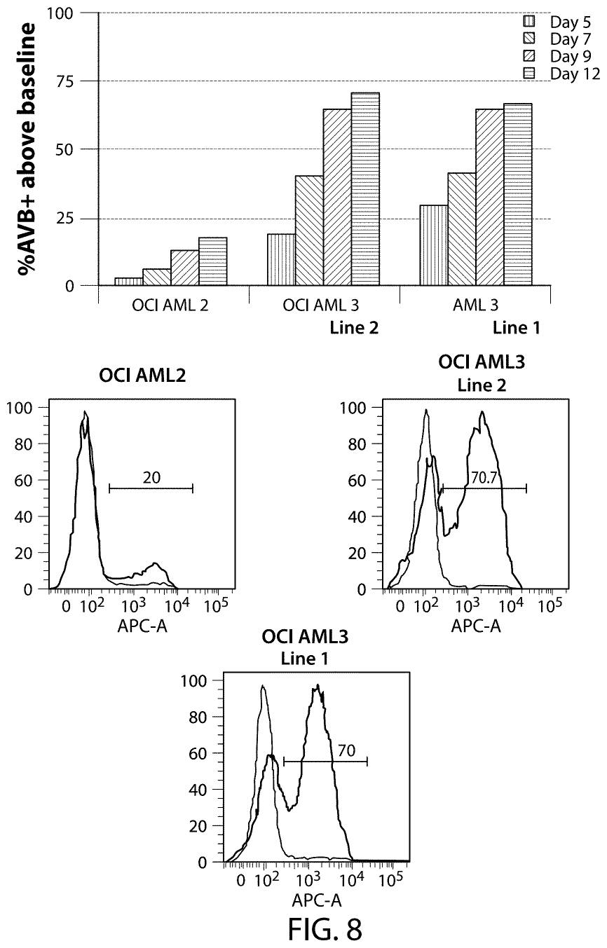

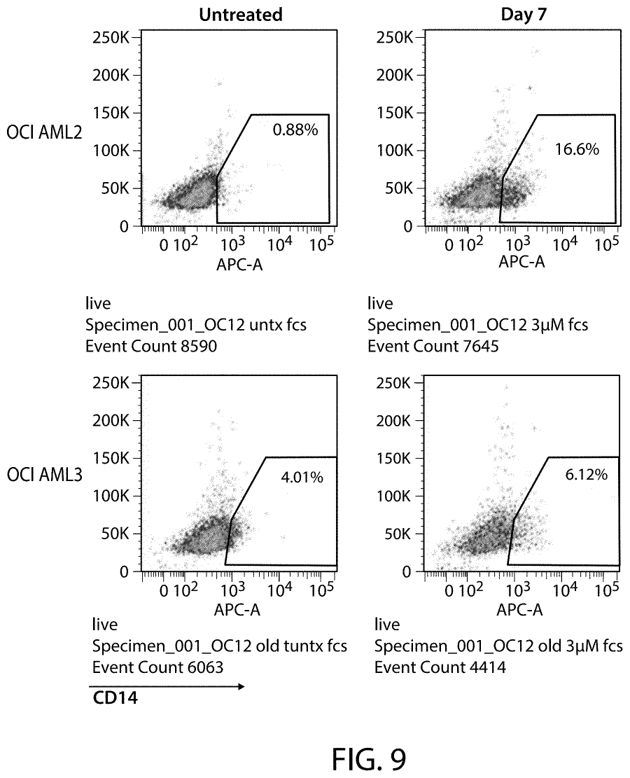

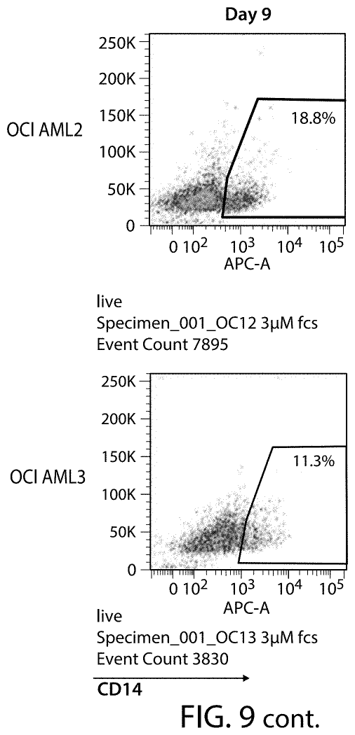

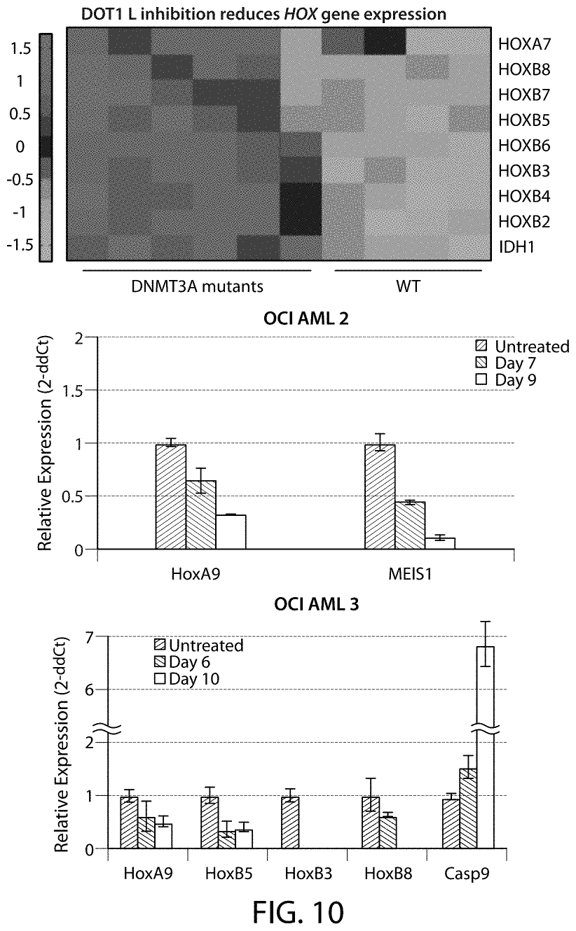

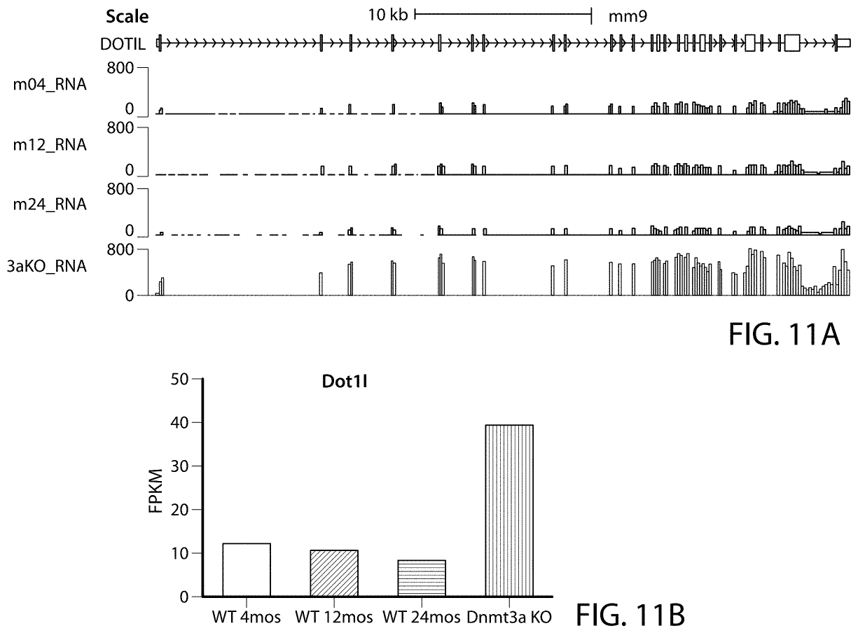

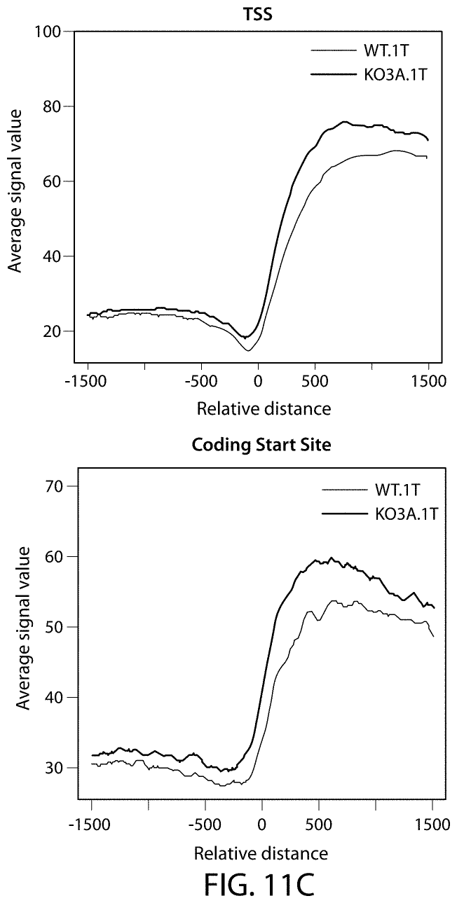

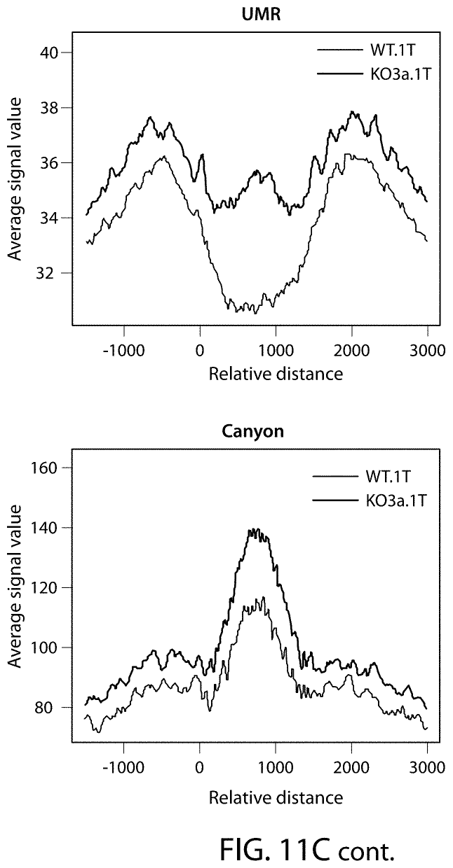

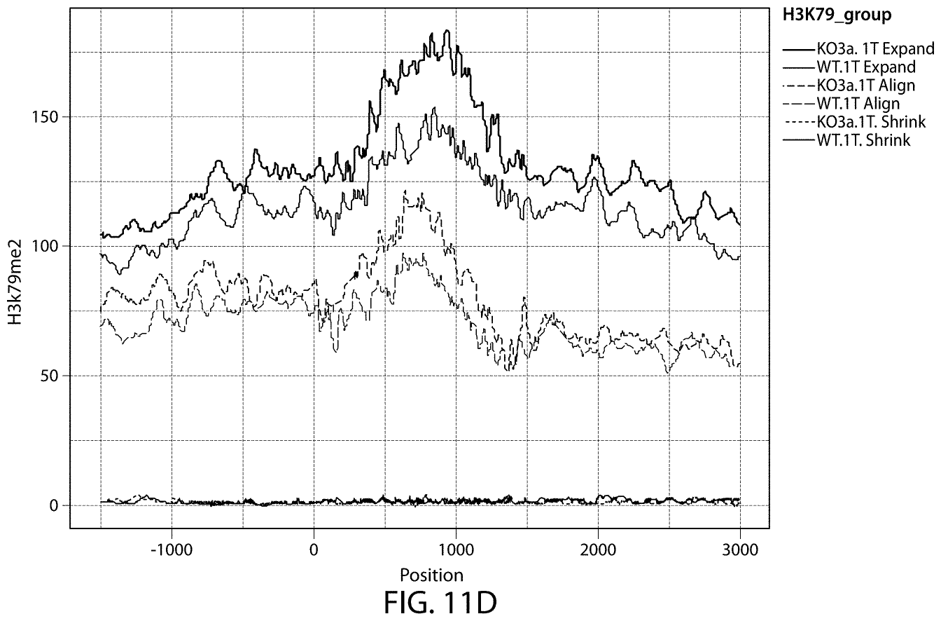

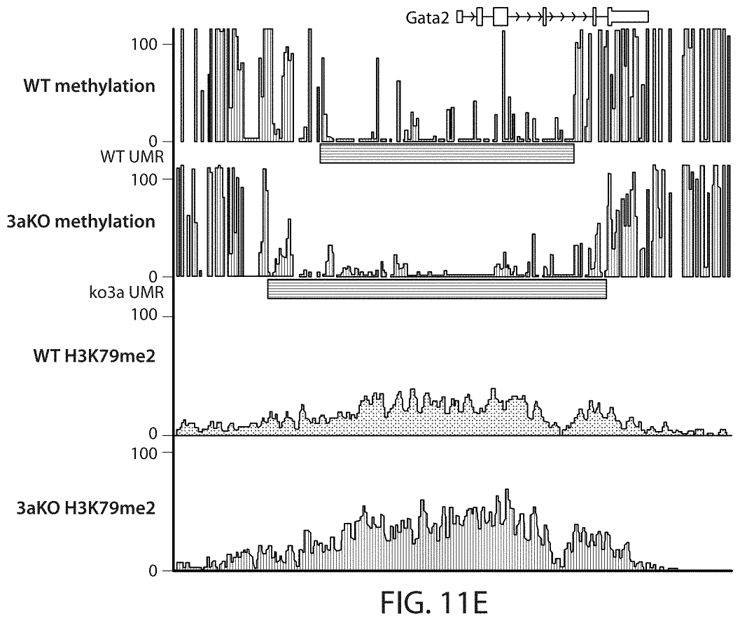

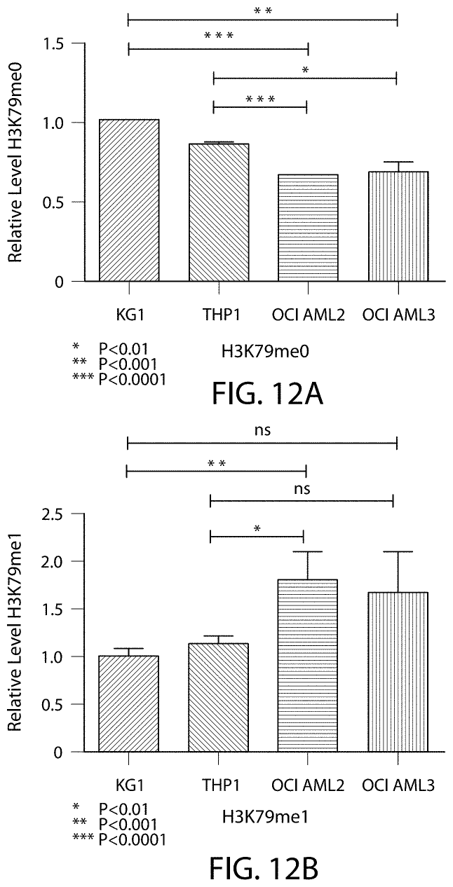

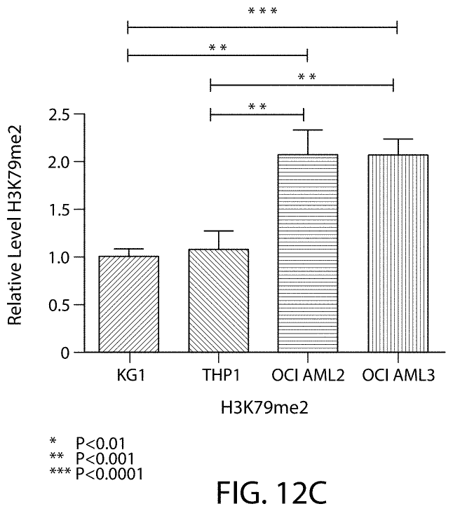

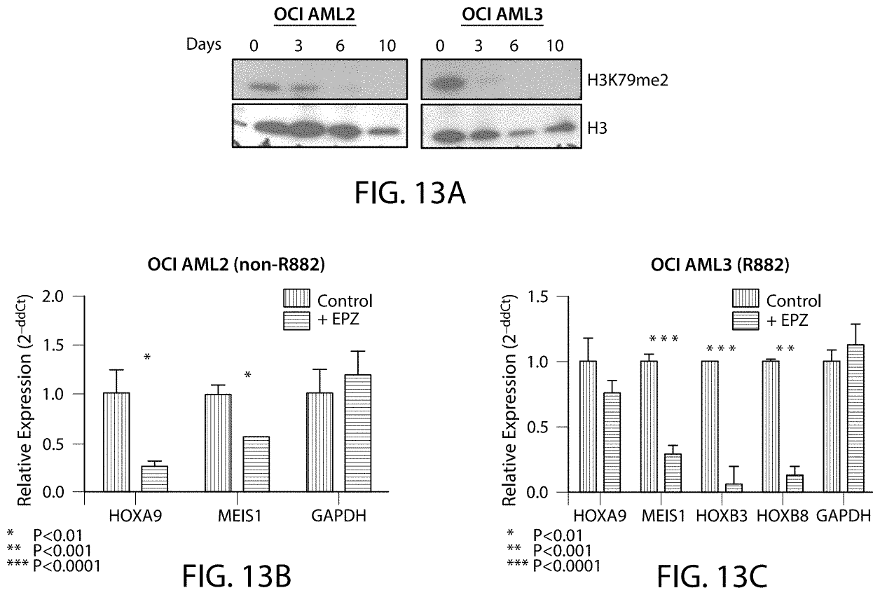

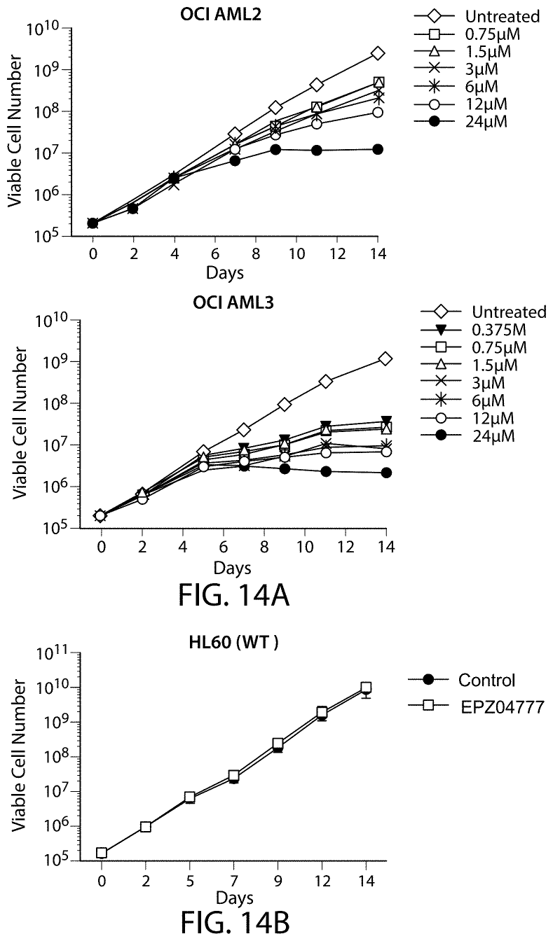

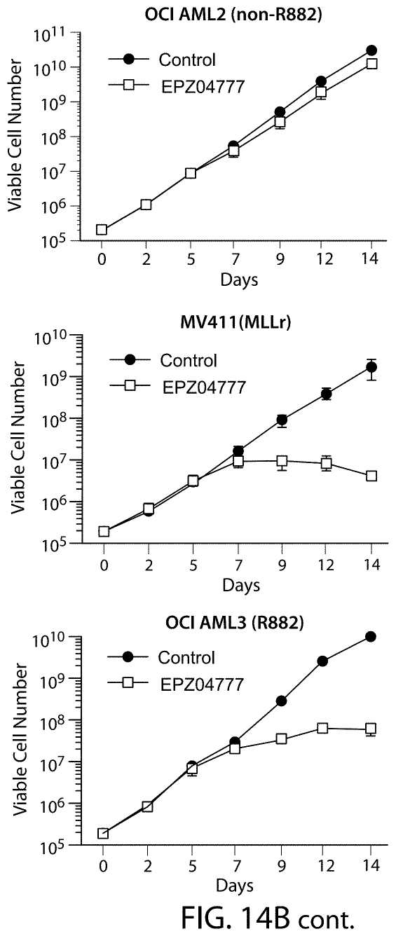

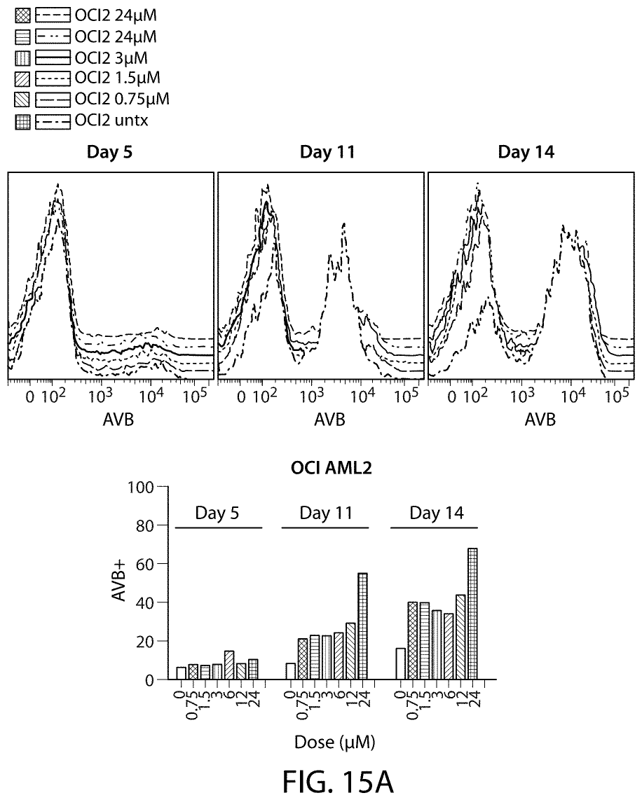

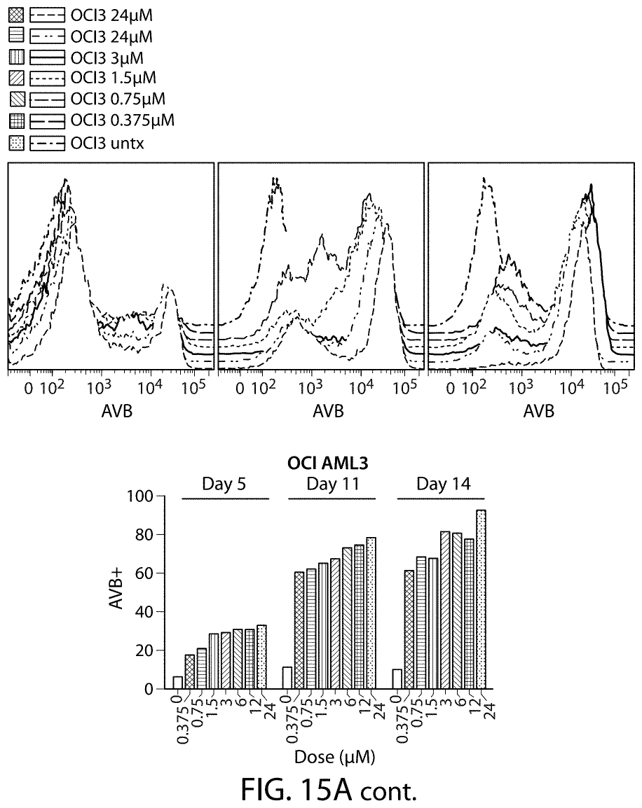

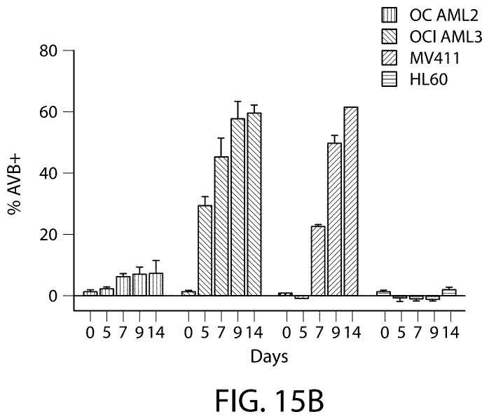

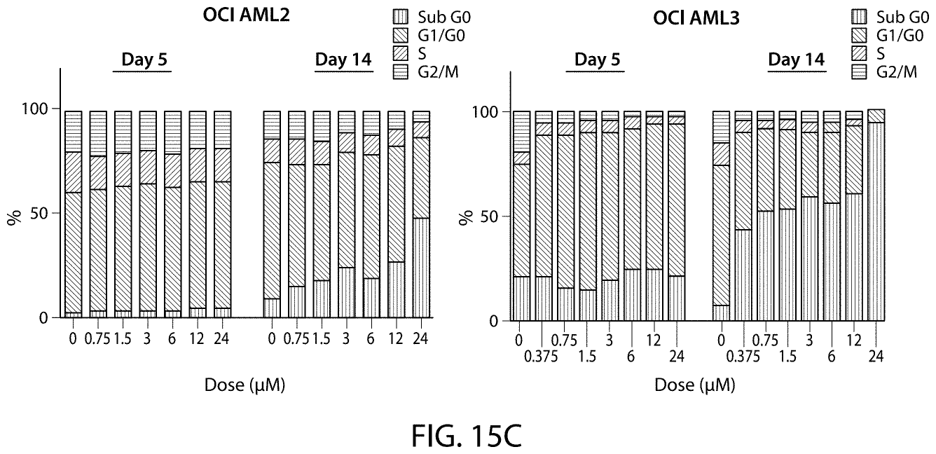

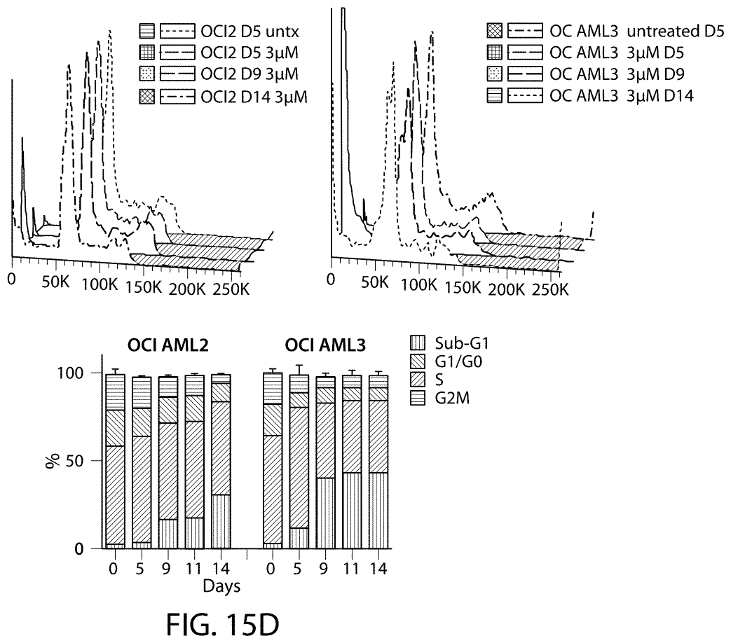

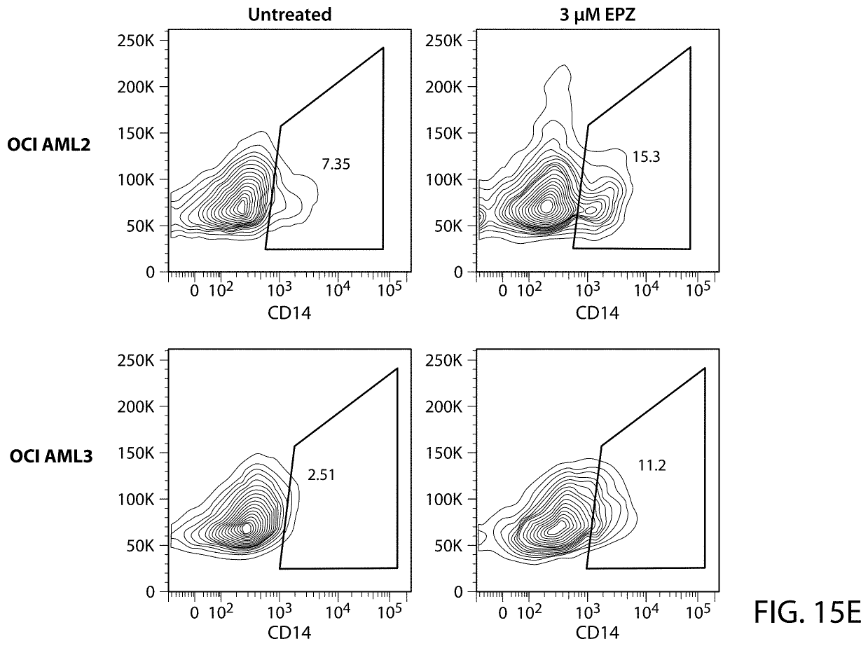

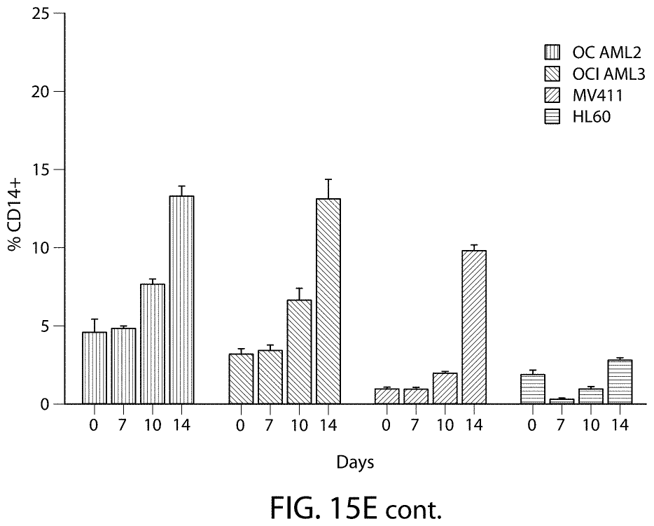

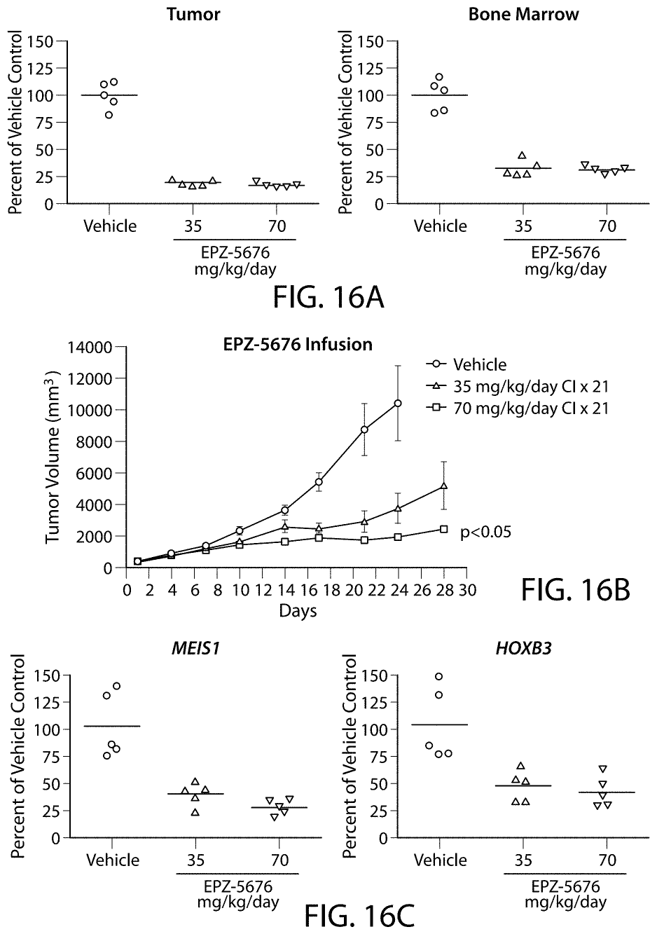

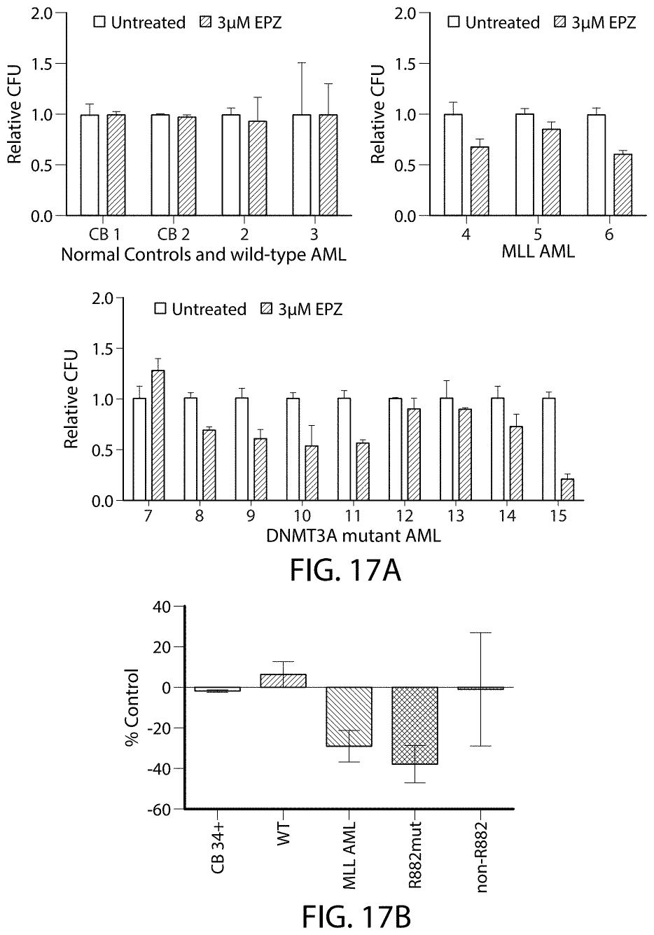

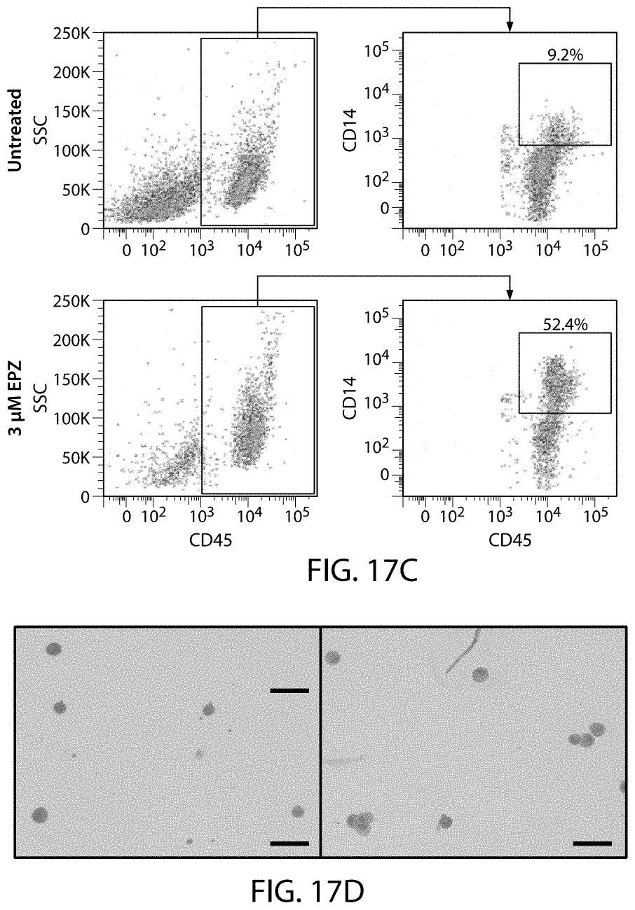

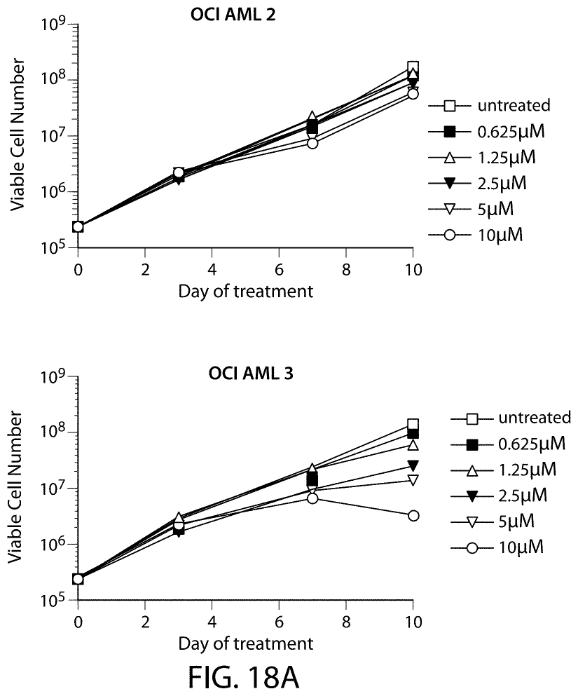

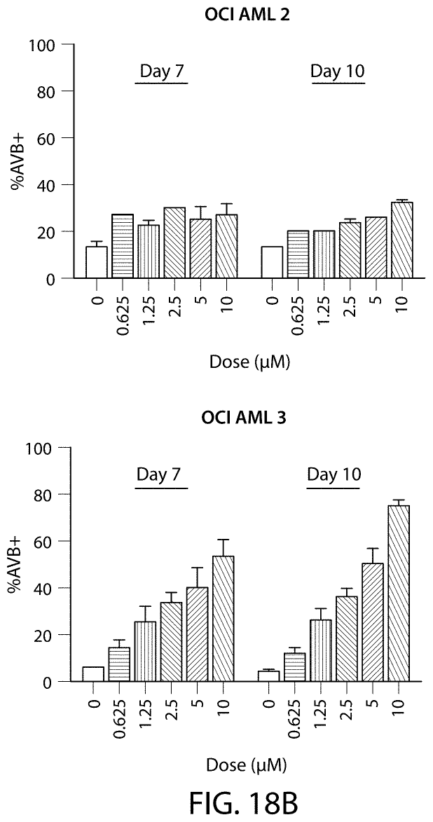

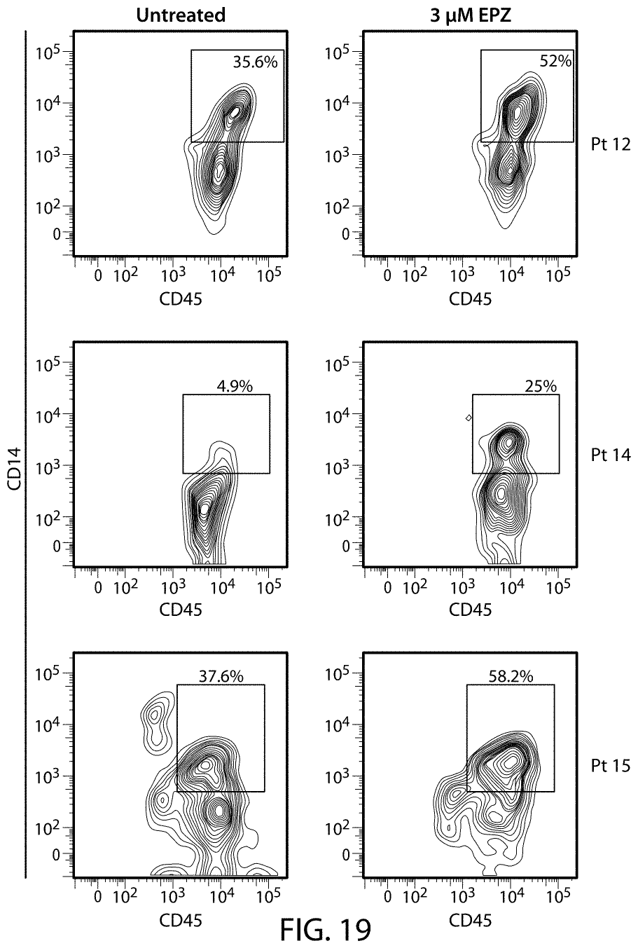

### DETAILED DESCRIPTION

Aspects of the present disclosure are based in part upon the surprising discovery that DOT1L inhibitors can effectively treat acute myeloid leukemia (AML) associated with one or more DNMT3A mutations.

In some embodiments, leukemia cells having one or more mutations in DNMT3A are sensitive to the DOT1L inhibitors as described herein. Accordingly, the present disclosure provides methods of treating, preventing, or alleviating one or more symptoms of leukemia associated with one or more mutations in DNMT3A in a subject by administering a therapeutically effective amount of a DOT1L inhibitor to the subject. In some embodiments, the present disclosure provides methods of treating, preventing, or alleviating one or more symptoms of leukemia in a subject having one or more expanded methylation canyons associated with DNMT3A mutation by administering a therapeutically effective amount of a DOT1L inhibitor to the subject. In some embodiments, the present disclosure provides methods of treating, preventing, or alleviating one or more symptoms of AML associated with one or more expanded methylation canyons in a subject as a result of DNMT3A mutation(s), wherein the expanded methylation canyons comprise HOX gene clusters, by administering a therapeutically effective amount of a DOT1L inhibitor to the subject. In some embodiments, the present disclosure provides methods of treating, preventing or alleviating one or more symptoms of AML associated with increased levels of H3K79me2. In some embodiments, increased levels of H3K79me2 result in H3K79me2 coating expanded methylation canyons associated with DNMT3A mutation(s).

As used herein, “methylation canyon” refers to a location in the genome of a subject (e.g., in cells of the subject, for example in hematopoietic stem cells of the subject) comprising expansive regions lacking DNA methylation (e.g., one or more hypomethylated DNA regions). In some embodiments, the methylation canyons described herein are greater than 3.5 kb in length and have a methylation ratio of less than 0.1. In some embodiments, the methylation canyons are enriched in genes that encode proteins involved in transcription regulation and/or in genes encoding a homeobox domain. In some embodiments, mutation(s) of DNMT3A cause(s) alterations in the size of methylation canyons. In some embodiments, mutation(s) of DNMT3A cause(s) the expansion of methylation canyons. In some embodiments, the expanded methylation canyons are coated with a higher amount of H3K79me2 than a corresponding cell that is positive for DNMT3A activity. In some embodiments, the expanding canyons coated with H3K79me2 comprise one or more HOX gene clusters.

Aspects of the disclosure are particularly useful for treating certain forms of AML that have a poor prognosis. Mutations of DNMT3A in AML typically predict a poor prognosis. According to aspects of the disclosure, mutations in DNMT3A induce an aggressive myeloid leukemia that can be treated with one or more DOT1L inhibitors.

In some embodiments, DOT1L inhibitor compounds described herein inhibit the histone methyltransferase activity of DOT1L or a mutant thereof and are useful to treat certain forms of AML. Based upon the surprising discovery that methylation regulation (e.g., histone methylation regulation) by DOT1L is involved in progression of AML bearing mutations in DNMT3A, the compounds described herein are useful for treating certain forms of acute myeloid leukemia.

In some embodiments, the present disclosure features a method for treating or alleviating one or more symptoms of DNMT3A-deficient AML (e.g., AML bearing one or more mutations in DNMT3A). The method includes administering to a subject in need thereof, a therapeutically effective amount of a DOT1L inhibitor or a pharmaceutically acceptable salt, prodrug, metabolite, polymorph, solvate, or stereoisomer thereof.

In some aspects, the disclosure relates to DNMT3A-deficient AML (e.g., AML bearing one or more mutations in DNMT3A). In some embodiments, DNMT3A-deficient AML is responsive to treatment with DOT1L inhibitors. In some embodiments, the present disclosure provides methods for the treatment of DNMT3A-deficient AML (e.g., to prevent or slow disease progression and/or to kill diseased cells) in a subject in need thereof by administering to a subject in need of such treatment, a therapeutically effective amount of a compound of the present disclosure (e.g., a DOT1L inhibitor), or a pharmaceutically acceptable salt, prodrug, metabolite, polymorph or solvate thereof. The present disclosure further provides the use of one or more DOT1L inhibitors, or a pharmaceutically acceptable salt, prodrug, metabolite, polymorph or solvate thereof, for the preparation of a medicament useful for the treatment of DNMT3A-deficient AML (e.g., in a human subject).

In some embodiments, the present disclosure provides methods for the treatment of a DNMT3A-deficient AML, the course of which is influenced by modulating the methylation status of histones or other proteins, wherein said methylation status is mediated at least in part by the activity of DOT1L.

Modulation of the methylation status of histones can in turn influence the level of expression of target genes activated by methylation, and/or target genes suppressed by methylation. The method includes administering to a subject in need of such treatment, a therapeutically effective amount of a DOT1L inhibitor as described herein, or a pharmaceutically acceptable salt, prodrug, metabolite, polymorph, solvate, or stereoisomer thereof.

In one aspect, methods described herein are useful to treat leukemia. In some embodiments, the leukemia is acute myeloid leukemia (AML). AML is a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with mutation(s) in DNMT3A (e.g., DNMT3A-deficient AML). Mutation of DNMT3A is known to occur at several locations of the gene. In some embodiments, mutations of the gene encoding DNMT3A cause a change in the amino acid sequence of the DNMT3A protein (UniProt Accession Number: Q9Y6K1 (SEQ ID NO:1)). Non-limiting examples of locations where DNMT3A mutations are known to occur are M315fs (frameshift), K468R, E477(stop), E505(stop), Q515(stop), G590(frameshift), Q615(stop), E616(frameshift), P718L, L723(frameshift), R729Q, R729W, R736H, A741V, L773(deletion), R792H, R8055, K829R, K841Q, R882P, R882H, R882C, and F909C. In some embodiments, other mutations are present in addition to mutations of DNMT3A. In some embodiments, other mutations co-occur with mutation of DNMT3A. In some embodiments, the co-occurring mutations are NPMc+, FLT3/ITD, IDH1/2, MLL PTD, and/or mutations of the cohesion complex.

In some aspects, mutations of DNMT3A are associated with types of cancer other than AML. Non-limiting examples of other cancers (e.g., other haem malignancies) associated with mutations of DNMT3A are Primary Myelo-Fibrosis, CMML, MDS, T-cell lymphoma, T-ALL, ETP-ALL, juvenile MML, mastocytosis, and MPAL.

The present disclosure further provides the use of a compound described herein, or a pharmaceutically acceptable salt, ester, prodrug, metabolite, polymorph or solvate thereof in the treatment of DNMT3A-deficient AML, or, for the preparation of a medicament useful for the treatment of such DNMT3A-deficient AML.

Compounds of the present disclosure can selectively inhibit proliferation of leukemia cells characterized by mutation(s) of DNMT3A.

Accordingly, the present disclosure provides methods for treating or alleviating a symptom of DNMT3A-deficient AML characterized by the presence of expanded methylation canyons associated with H3K79me2 by a compound of the present disclosure, or a pharmaceutically acceptable salt, ester, prodrug, metabolite, polymorph or solvate thereof.

The present disclosure also provides methods for treating or alleviating a symptom of DNMT3A-deficient AML characterized by the presence of expanded methylation canyons associated with H3K79me2. For example, in some embodiments a method comprises obtaining sample from a subject, detecting the presence of one or more mutations in DNMT3A in the sample, and when mutation is present in the sample, administering to the subject a therapeutically effective amount of a DOT1L inhibitor

In other aspects, the present disclosure provides personalized medicine, treatment and/or AML management for a subject by genetic screening for mutations in DNMT3A in the subject. For example, the present disclosure provides methods for treating, preventing or alleviating a symptom of leukemia or a precancerous condition by determining responsiveness of the subject to a DOT1L inhibitor and when the subject is responsive to the DOT1L inhibitor, administering to the subject a therapeutically effective amount of the DOT1L inhibitor, or a pharmaceutically acceptable salt, prodrug, metabolite, polymorph, solvate, or stereoisomer thereof. In some embodiments, the responsiveness can be determined by obtaining a sample from the subject and detecting one or more mutations in DNMT3A, and the presence of such mutations indicates that the subject is responsive to the DOT1L inhibitor. Once the responsiveness of a subject is determined, a therapeutically effective amount of a DOT1L inhibitor can be administered. The therapeutically effective amount of a DOT1L inhibitor can be determined by one of ordinary skill in the art.

In other aspects, the present disclosure provides personalized medicine, treatment and/or cancer management for a subject by genetic screening of methylation canyons. In some aspects, AML subtypes are associated with particular expanded methylation canyons, including expanded methylation canyons coated with a higher amount of H3K79me2 than a corresponding cell that is positive for DNMT3A activity. In some aspects, the expanded methylation canyons coated with H3K79me2 comprise HOX gene clusters.

As used herein, the term “responsiveness” is interchangeable with terms “responsive”, “sensitive”, and “sensitivity”, and it is meant that a subject shows one or more therapeutic responses when administered an DOT1L inhibitor, e.g., leukemia cells or leukemia progenitor cells of the subject undergo apoptosis and/or necrosis, differentiation and/or display reduced growth, division, or proliferation. This term can also mean that a subject will or has a higher probability, relative to the population at large, of having a therapeutic response when administered an DOT1L inhibitor, e.g., leukemia cells or leukemia progenitor cells of the subject undergo apoptosis and/or necrosis, differentiation and/or display reduced growth, division, or proliferation.

As used herein, a “subject” is interchangeable with a “subject in need thereof”, both of which refers to a subject having DNMT3A-deficient AML that involves DOT1L-mediated protein methylation, or a subject having an increased risk of developing such a disorder relative to the population at large. A subject in need thereof may be a subject having a DNMT3A-deficient AML. A subject in need thereof can have a precancerous condition. In some embodiments, a subject in need thereof has leukemia. A subject in need thereof can have leukemia associated with DOT1L, for example AML. A subject in need thereof can have AML associated with one or more mutations in DNMT3A. A subject in need thereof can have DNMT3A-deficient AML associated with expanded methylation canyons associated with H3K79me2.

As used herein, a “subject” includes a mammal. The mammal can be, e.g., a human or appropriate non-human mammal, such as a primate, mouse, rat, dog, cat, cow, horse, goat, camel, sheep or a pig. The subject can also be a bird or fowl. In one embodiment, the mammal is a human. A subject can be male or female.

A subject in need thereof can be one who has been previously diagnosed or identified as having leukemia or a precancerous condition. A subject in need thereof can also be one who is having (suffering from) leukemia or a precancerous condition. Alternatively, a subject in need thereof can be one who has an increased risk of developing such disorder relative to the population at large (e.g., a subject who is predisposed to developing such disorder relative to the population at large).

Optionally a subject in need thereof has already undergone, is undergoing or will undergo, at least one therapeutic intervention for the leukemia or precancerous condition prior to the treatment with a DOT1L inhibitor.

A subject in need thereof may have refractory leukemia after their most recent therapy.

“Refractory leukemia” means leukemia that does not respond to treatment. The leukemia may be resistant at the beginning of treatment or it may become resistant during treatment.

Refractory leukemia is also called resistant leukemia. In some embodiments, the subject in need thereof has leukemia recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary leukemia as a result of a previous therapy. “Secondary leukemia” means leukemia that arises after, due to, or as a result from previous carcinogenic therapies, such as chemotherapy. In some embodiments, the secondary leukemia is AML. In some embodiments, the secondary leukemia is DNMT3A-deficient AML.

In some embodiments, the mutations and/or chromosomal alterations referred to herein are somatic mutations or alterations. The term “somatic” mutation or alteration refers to a mutation or alteration (e.g., deleterious) in at least one gene allele (e.g., one or both alleles or copies of a chromosomal region) that is not found in every cell of the body, but is found only in isolated cells. A characteristic of the somatic changes as used herein is, that they are restricted to particular tissues or even parts of tissues or cells within a tissue and are not present in the whole organism harboring the tissues or cells. The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene.

Accordingly, an increase in mRNA or protein expression and/or activity levels can be detected using any suitable method available in the art. For example, an increase in activity level can be detected by measuring the biological function of a gene product (e.g., activity of DOT1L) or transcriptional activity (e.g., expression levels of target genes can be assayed using RT-PCR or other suitable technique). In some embodiments, genetic modifications (e.g., one or more mutations of DNMT3A) are associated with expanded methylation canyons and may be detected by a sequencing analysis. In some embodiments, expanded methylation canyons may be detected by methods including, but not limited to, whole genome bisulfite sequencing, methylation-specific-PCR, or Methylated DNA immunoprecipitation (MeDIP). Methods can entail the steps of genomic DNA purification, PCR amplification to amplify the region of interest, cycle sequencing, sequencing reaction cleanup, capillary electrophoresis, and/or data analysis. Alternatively or in addition, a method may include the use of microarray-based targeted region genomic DNA capture and/or sequencing.

In some embodiments, levels of H3K79me2 can be determined by histone extraction followed by Western blotting or bisulfite sequencing. However, it should be appreciated that other techniques can be used.

Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, quantitative RT-PCR, expression array or RNA-sequencing. Detection of polypeptide expression (e.g., wild-type or mutant) can be carried out with any suitable immunoassay in the art, such as Western blot analysis.

By “sample” is meant any biological sample derived from the subject, includes but is not limited to, cells, tissues samples, body fluids (including, but not limited to, mucus, blood, plasma, serum, urine, saliva, and semen), cancer cells, and cancer tissues. In some embodiments, the sample is selected from bone marrow, peripheral blood cells, blood, cerebrospinal fluid, skin lesions, chloroma biopsies, plasma and serum. In some embodiments, a sample consists of or contains leukemic blasts from a patient that has a hematologic malignancy.

Samples can be provided by the subject under treatment or testing. Alternatively samples can be obtained by the physician according to routine practice in the art.

The present disclosure also provides methods for determining predisposition of a subject to DNMT3A-deficient AML by obtaining a sample from the subject and detecting one or more mutations in DNMT3A. In some embodiments, the presence of such mutations can be used to indicate that the subject is predisposed to (e.g., has a higher risk of) developing leukemia compared to a subject without such mutations.

The term “predisposed” as used herein in relation to leukemia or a precancerous condition is to be understood to mean the increased probability (e.g., at least 1%, 5%, 10%, 20%, 30%, 40%, 50%, 60%, 70%, 80%, 90%, 100%, 150%, 200%, or more increase in probability) that a subject with one or more mutations in DNMT3A, will suffer leukemia, as compared to the probability that another subject not having one or more mutations in DNMT3A, will suffer leukemia, under circumstances where other risk factors (e.g., chemical/environment, food, and smoking history, etc.) for having leukemia between the subjects are the same.

“Risk” in the context of the present disclosure, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

In other example, the present disclosure provides methods of AML management in a subject by determining predisposition of the subject to DNMT3A-deficient AML periodically. The methods comprise steps of obtaining a sample from the subject and detecting one or more mutations in DNMT3A, and the presence of such mutation(s) indicates that the subject is predisposed to developing DNMT3A-deficient AML compared to a subject without such mutations in DNMT3A.

As used herein, the term “acute myeloid leukemia (AML)” refers to a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with mutations in DNMT3A (DNMT3A-deficient AML). In some aspects, AML subtypes are associated with the presence of particular methylation canyons. In some aspects, the expanded methylation canyons are coated with a higher amount of H3K79me2 than a corresponding cell that is positive for DNMT3A activity. In some embodiments, the expanded methylation canyons associated with H3K79me2 comprise HOX gene clusters.

As used herein, “treating” or “treat” describes the management and care of a patient for the purpose of combating a disease, condition, or disorder and includes the administration of a compound of the present disclosure, or a pharmaceutically acceptable salt, prodrug, metabolite, polymorph or solvate thereof, to alleviate the symptoms or complications of a disease, condition or disorder, or to eliminate the disease, condition or disorder.

A compound of the present disclosure, or a pharmaceutically acceptable salt, prodrug, metabolite, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as leukemia, which can occur in multiple locations, is alleviated if the severity of the leukemia is decreased within at least one of multiple locations.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals. As used herein the term “sign” is also defined as an indication that something is not right in the body. But signs are defined as things that can be seen by a doctor, nurse, or other health care professional.

Treating or preventing a leukemia can result in a reduction in the rate of leukemia cell or leukemia progenitor cell proliferation. Preferably, after treatment, the rate of leukemia-associated cell proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in an increase in the rate of normal blood cell proliferation. Preferably, after treatment, the rate of normal blood cell proliferation is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in a reduction in the proportion of proliferating leukemia cells or leukemia progenitor cells. Preferably, after treatment, the proportion of proliferating leukemia cells or leukemia progenitor cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a leukemia can result in an increase in the proportion of normal blood cells. Preferably, after treatment, the proportion of proliferating normal cells is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating normal cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing leukemia can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of excessive accumulation of immature cells (blasts) and differentiation arrest, or disordered (dysplastic) differentiation.

Treating leukemia, for example AML, can result in leukemia cell death, and preferably, leukemia cell death results in a decrease of at least 10% in number of leukemia cells in a population. More preferably, leukemia cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, leukemia cell death occurs by apoptosis.

Treating leukemia, for example AML, can result in leukemia cell differentiation, and preferably, leukemia cell differentiation results in a decrease of at least 10% in number of undifferentiated leukemia cells (leukemic blasts) in a population. More preferably, leukemia cell differentiation means a decrease of at least 20%; more preferably, a decrease of at least 30%; more preferably, a decrease of at least 40%; more preferably, a decrease of at least 50%; most preferably, a decrease of at least 75%. The number of cells in a population may be measured by any reproducible means. The number of blasts and differentiated cells in a population can be measured by fluorescence activated cell sorting (FACS), immunofluorescence microscopy and light microscopy.

In some embodiments, an effective amount of a compound of the present disclosure, or a pharmaceutically acceptable salt, prodrug, metabolite, polymorph or solvate thereof, is not significantly cytotoxic to normal cells. A therapeutically effective amount of a compound is not significantly cytotoxic to normal cells if administration of the compound in a therapeutically effective amount does not induce normal cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present disclosure, or a pharmaceutically acceptable salt, prodrug, metabolite, polymorph or solvate thereof, can induce or activate cell death selectively in AML cells. Administering to a subject in need thereof a compound of the present disclosure, or a pharmaceutically acceptable salt, prodrug, metabolite, polymorph or solvate thereof, can induce or activate cell death selectively in AML cells. Contacting a cell with a compound of the present disclosure, or a pharmaceutically acceptable salt, prodrug, metabolite, polymorph or solvate thereof, can induce cell death selectively in one or more cells affected by AML. Preferably, administering to a subject in need thereof a compound of the present disclosure, or a pharmaceutically acceptable salt, prodrug, metabolite, polymorph or solvate thereof, induces cell death selectively in one or more cells affected by AML.

In some embodiments, the present disclosure relates to a method of treating or preventing AML by administering a compound of the present disclosure, or a pharmaceutically acceptable salt, prodrug, metabolite, polymorph or solvate thereof, to a subject in need thereof, where administration of the compound of the present disclosure, or a pharmaceutically acceptable salt, prodrug, metabolite, polymorph or solvate thereof, results in one or more of the following: accumulation of cells in G1 and/or S phase of the cell cycle, cytotoxicity via cell death in AML cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N. Y.; Enna et al., Current Protocols in Pharmacology, John Wiley & Sons, N. Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

As used herein, a DOT1L inhibitor is an inhibitor of DOT1L-mediated protein methylation (e.g., an inhibitor of histone methylation). In some embodiments, a DOT1L inhibitor is a small molecule inhibitor of DOT1L. In some embodiments, a DOT1L inhibitor is a compound of formula:

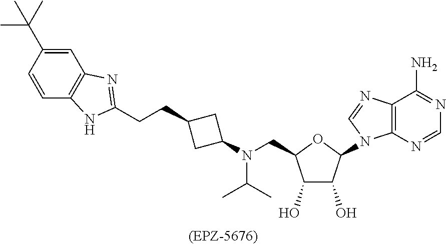 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof.

In some embodiments, a DOT1L inhibitor is a compound of formula:

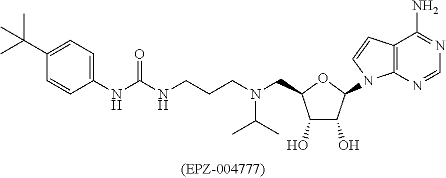 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof.

In some embodiments, a DOT1L inhibitor is a compound of formula:

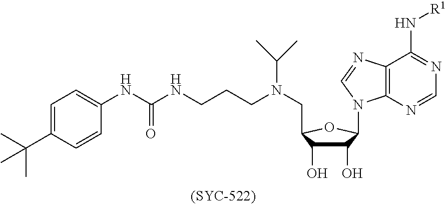

wherein R1 is a H, or a pharmaceutically acceptable salt, prodrug, metabolite, polymorph, solvate or stereoisomer thereof.

Other DOT1L inhibitors suitable for use according to methods described herein are provided in WO2012/075381, WO2012/075492, WO2012/082436, WO2012/75500, WO2014/026198, WO2014/035140, US2014/0100184, and in J. Med Chem. (2013), 56: p. 8972-8983, the contents of each of which are hereby incorporated by reference in their entirety. The activity of a DOT1L inhibitor can be evaluated in an assay, for example by comparing the histone methyltransferase activity of DOT1L (e.g., methylation of histone substrates such as H3K79 by immunoblot) in the presence or absence of different amounts of the inhibitor.

The disclosure also relates to a pharmaceutical composition of a therapeutically effective amount of a DOT1L inhibitor disclosed herein and a pharmaceutically acceptable carrier.

The disclosure also relates to a pharmaceutical composition of a therapeutically effective amount of a salt of a DOT1L inhibitor disclosed herein and a pharmaceutically acceptable carrier.

The disclosure also relates to a pharmaceutical composition of a therapeutically effective amount of a hydrate of a DOT1L inhibitor disclosed herein and a pharmaceutically acceptable carrier.

The present disclosure also relates to use of the compounds disclosed herein in preparation of a medicament for treating or preventing leukemia. The use includes a DOT1L inhibitor disclosed herein for administration to a subject in need thereof in a therapeutically effective amount. The leukemia can be AML. In some embodiments, the AML is DNMT3A-deficient AML. In some embodiments, the DNMT3A-deficient AML is associated with one or more expanded methylation canyons. In some embodiments, the expanded methylation canyons are coated with a higher amount of H3K79me2 than a corresponding cell that is positive for DNMT3A activity. In some embodiments, the expanded methylation canyons coated with H3K79me2 comprise HOX gene clusters.

In some embodiments, compounds provided herein can be formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered continuously (e.g., intravenously) three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations). In some embodiments the administration regimen is a continuous IV infusion (e.g., 24 hours per day) for one or more weeks (e.g., 1-4, 4-8, or longer), for example a 28-day continuous IV infusion of each 28-day cycle.

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It should be appreciated that in some embodiments, a DOT1L inhibitor compound or composition can be administered as a monotherapy. As used herein, “monotherapy” refers to the administration of a single active or therapeutic compound to a subject in need thereof. In some embodiments, monotherapy will involve administration of a therapeutically effective amount of a single active compound, for example, AML monotherapy with one of the DOT1L inhibitor compounds described herein, or a pharmaceutically acceptable salt, prodrug, metabolite, analog or derivative thereof, to a subject in need of treatment of AML. In one aspect, the single active DOT1L inhibitor compound is a compound described herein, or a pharmaceutically acceptable salt, prodrug, metabolite, polymorph or solvate thereof.

It will be appreciated that in some embodiments, two or more DOT1L inhibitor compounds can be administered to a subject (e.g., to treat AML).

It also will be appreciated that in some embodiments one or more DOT1L inhibitor compounds or compositions, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

In some embodiments, a DOT1L inhibitor compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is an AML standard of care agent. In certain embodiments, an additional therapeutically active agent is Ara-C, or daunorubicin. In certain embodiments, an additional therapeutically active agent is a DNA methyltransferase inhibitor. In certain embodiments, an additional therapeutically active agent is azacitidine or decitabine. In certain embodiments, an additional therapeutically active agent is a histone deacetylase inhibitor. In certain embodiments, an additional therapeutically active agent is vorinostat or panobinostat. In certain embodiments, an additional therapeutically active agent is a demethylase inhibitor. In certain embodiments, an additional therapeutically active agent is tranylcypromine or LSD1 inhibitor II. In certain embodiments, an additional therapeutically active agent is a bromodomain inhibitor. In certain embodiments, an additional therapeutically active agent is IBET-151 or JQ1. In certain embodiments, an additional therapeutically active agent is an ALL standard of care agent. In certain embodiments, an additional therapeutically active agent is mitoxantrone, methotrexate, mafosfamide, prednisolone, or vincristine.

In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

It also should be appreciated that in some embodiments, a DOT1L inhibitor compound or composition can be administered in conjunction with chemotherapy, radiation therapy, and/or a cytostatic agent. In some embodiments, treatment methods described herein are administered in conjunction with anti-VEGF or anti-angiogenic factor, and/or p53 reactivation agent. Non-limiting examples of cancer chemotherapeutic agents include, but are not limited to, irinotecan (CPT-11); erlotinib; gefitinib (Iressa®); imatinib mesylate (Gleevec®); oxalipatin; anthracyclins-idarubicin and daunorubicin; doxorubicin; alkylating agents such as melphalan and chlorambucil; cis-platinum, methotrexate, and alkaloids such as vindesine and vinblastine. A cytostatic agent is any agent capable of inhibiting or suppressing cellular growth and multiplication. Non-limiting examples of cytostatic agents include paclitaxel, 5-fluorouracil, 5-fluorouridine, mitomycin-C, doxorubicin, and zotarolimus. Other cancer therapeutics that can be used in conjunction with a DOT1L inhibitor include inhibitors of matrix metalloproteinases such as marimastat, growth factor antagonists, signal transduction inhibitors and protein kinase C inhibitors. In some embodiments, methods described herein can be used in combination with treatment options such immunotherapy and/or cancer vaccines.

It should be appreciated that in some embodiments, the term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies.

The present disclosure also provides pharmaceutical compositions comprising one or more DOT1L inhibitor compounds described herein, and optionally one or more additional agents described herein, in combination with at least one pharmaceutically acceptable excipient or carrier.

A “pharmaceutical composition” is a formulation containing one or more DOT1L inhibitor compounds in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition described herein can be administered to a subject in many of the well-known methods currently used for chemotherapeutic treatment. For example, for treatment of leukemia, a DOT1L inhibitor compound or formulation may be injected directly into the blood stream or body cavities or taken orally or applied through the skin with patches. The dose chosen should be sufficient to constitute effective treatment but not as high as to cause unacceptable side effects. The state of the disease condition (e.g., leukemia, for example, AML) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In some embodiments, the disease or condition to be treated is leukemia (e.g., AML, for example DNMT3A-deficient AML).

For a DOT1L inhibitor compound or formulation, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms described herein are dictated by and directly dependent on the unique characteristics of the active compound and the particular therapeutic effect to be achieved.

In therapeutic applications, the dosages of the pharmaceutical compositions used as described herein vary depending on the agent or combination of agents, the age, weight, and clinical condition of the recipient patient, and the experience and judgment of the clinician or practitioner administering the therapy, among other factors affecting the selected dosage. Generally, the dose should be sufficient to result in slowing, and preferably regressing, the proliferation of leukemia cells and also preferably causing complete regression of the leukemia. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m, and age in years). An effective amount of a pharmaceutical agent is that which provides an objectively identifiable improvement as noted by the clinician or other qualified observer. For example, regression of leukemia in a patient may be measured with reference to the number of leukemia cells or leukemia precursor cells. Decrease in the number of leukemia cells indicates regression. Regression is also indicated by failure of leukemia cells to reoccur after treatment has stopped. As used herein, the term “dosage effective manner” refers to amount of an active compound to produce the desired biological effect in a subject or cell.

The compounds of the present disclosure are capable of further forming salts.

As used herein, “pharmaceutically acceptable salts” refer to derivatives of the compounds described herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds described herein can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds described herein can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H. , Design of Prodrugs, p1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds can be found in Remington: the Science and Practice of Pharmacy, 19th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing aspects of the present disclosure. The examples do not limit the claimed invention. Based on the present disclosure the skilled artisan can identify and employ other components and methodology useful for practicing aspects of the present invention.

For the compounds described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers.

The pharmaceutical compositions can be included in a container, pack, or dispenser together with instructions for administration. Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the invention remains operable.

Moreover, two or more steps or actions can be conducted simultaneously.

Compounds suitable for the methods of the disclosure, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

1. A method of treating acute myeloid leukemia (AML), the method comprising administering a composition comprising a DOT1L inhibitor to a subject having AML associated with one or more DNMT3A mutations.

2. The method of claim 1, wherein the one or more DNMT3A mutations cause a dominant negative loss of function of DNMT3A activity.

3. The method of claim 1, wherein a biological sample from the subject is identified as having one or more expanded DNA methylation canyons.

4. The method of claim 3, wherein the one or more expanded DNA methylation canyons comprise a HOX gene cluster.

5. The method of claim 4, wherein the HOX gene cluster is a HOXA gene cluster.

6. The method of claim 4, wherein the HOX gene cluster is a HOXB gene cluster.

7. The method of claim 1, wherein a biological sample from the subject is identified as having a level of H3K79me2 that is higher than in a DNMT3A wild-type control.

8. A method of treating a subject having AML, the method comprising:
a. obtaining a biological sample from the subject;
b. detecting the presence of one or more DNMT3A mutations in the biological sample, detecting the presence of one or more expanded DNA methylation canyons in the biological sample, or detecting the presence of a higher level of H3K79me2 in the biological sample than in a DNMT3A wild-type control; and,
c. administering to the subject a composition comprising a DOT1L inhibitor.

9.-10. (canceled)

11. A method of identifying a subject having AML that is responsive to treatment with a DOT1L inhibitor, the method comprising:
a. obtaining a biological sample from the subject;
b. assaying the biological sample for the presence of one or more DNMT3A mutations, assaying the biological sample for the presence of one or more expanded DNA methylation canyons, or assaying a level of H3K79me2 in the biological sample; and,
c. identifying the subject as responsive to treatment with a DOT1L inhibitor if one or more DNMT3A mutations are detected in the biological sample, if one or more expanded DNA methylation canyons are detected in the biological sample, or if the level of H3K79me2 is higher than in a DNMT3A wild-type control.

12.-13. (canceled)

14. The method of claim 1, wherein the DOT1L inhibitor is a compound of formula: 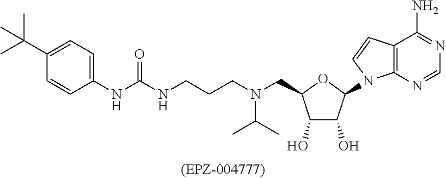 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]
or a pharmaceutically acceptable salt thereof.

15. The method of claim 1, wherein the DOT1L inhibitor is a compound of formula: 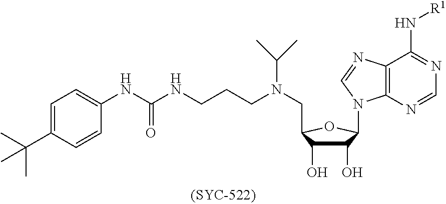
wherein R1 is a H, or a pharmaceutically acceptable salt thereof.

16. The method of claim 1, wherein the DOT1L inhibitor is a compound of formula: 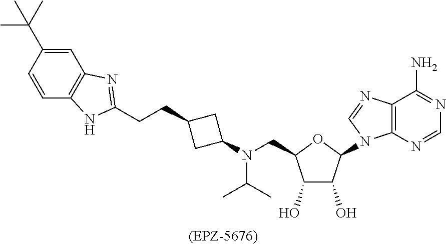 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]
or a pharmaceutically acceptable salt thereof.

17. The method of claim 8, wherein the biological sample is selected from the group consisting of bone marrow, peripheral blood cells, blood, cerebrospinal fluid, skin lesions, chloroma biopsy, plasma, serum, urine, saliva and a cell.

18. The method of claim 1, wherein the presence of one or more mutations of DNMT3A are detected by genome sequence analysis, next generation sequencing, and/or PCR-based mutation detection.


SEQUENCE LISTING (1 sequence)

SEQ ID NO: 1 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV